(19) United States
(12) Patent Application Publication
(10) Pub. No.: US 2023/0381193 A1
(43) Pub. Date: Nov. 30, 2023

(54) METHODS FOR THE TREATMENT OF CHILDHOOD-ONSET FLUENCY DISORDER

(71) Applicant: Noema Pharma AG, Basel (CH)

(72) Inventor: George Garibaldi, Basel (CH)

(21) Appl. No.: 18/360,533

(22) Filed: Jul. 27, 2023

Related U.S. Application Data
(63) Continuation of application No. PCT/EP2022/052131, filed on Jan. 28, 2022.
(60) Provisional application No. 63/142,876, filed on Jan. 28, 2021.
(60) Provisional application No. 63/196,902, filed on Jun. 4, 2021.

(51) Int. Cl. A61K 31/5377; A61K 31/5513; A61K 9/48; A61P 3/08

(52) U.S. Cl. CPC A61K 31/5377; A61K 31/5513; A61K 9/4816; A61P 3/08

(57) ABSTRACT

Provided herein are methods of treating Childhood-Onset Fluency Disorder (COFD) in a subject in need thereof by administering to the subject compositions comprising a PDE10A inhibitor. Also disclosed are methods of treating COFD in a subject in need thereof by administering to the subject compositions comprising the compound of Formula I below:

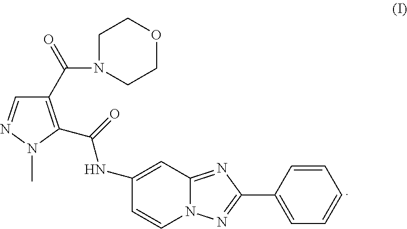 [SMILES: Cn1ncc(C(=O)N2CCOCC2)c1C(=O)Nc1ccn2nc(-c3ccccc3)nc2c1]

### RELATED APPLICATIONS

This application is a continuation of International Application Number PCT/EP2022/052131 filed on Jan. 28, 2022, which claims priority to U.S. Provisional Patent Application Nos. 63/142,876, and 63/196,902, respectively filed on Jan. 28, 2021, and Jun. 4, 2021; the contents of each of which are hereby incorporated by reference for all purposes.

### BACKGROUND

Childhood-Onset Fluency Disorder (COFD; aka stuttering or development stuttering or stammer) is a neuro-developmental speech disorder that involves frequent and significant problems with normal fluency and flow of speech. COFD, typically characterized by recurrent prolongations, reverberations or blocs of sounds, syllables, phrases or words, can lead to significant secondary effects, including negative self-perception and negative perception by others, anxiety, and occasionally depression. It affects about 5% to 10% of preschoolers and about 1% of adults. (R. W. Sander, et al. American Family Physician, 2019, 100(9): 556-560).

There are currently no approved pharmacological treatments for COFD. Disorder management include speech therapy, psychotherapy, and antipsychotics, but with limited effectiveness. Antipsychotics are often used off label and are associated with serious side effects.

Therefore, there is an unmet medical need to develop new methods that can effectively treat COFD without serious side effects.

### SUMMARY

The disclosure is directed to, in one aspect, a method of treating Childhood-Onset Fluency Disorder (COFD), wherein the method comprises administering to a subject in need thereof a composition containing a therapeutically effective amount of a phosphodiesterase 10A (PDE10A) inhibitor or a pharmaceutically acceptable salt thereof. In another aspect, a method of treating Childhood-Onset Fluency Disorder (COFD), wherein the method comprises administering to a subject in need thereof a composition containing a therapeutically effective amount of a phosphodiesterase 10A (PDE10A) inhibitor (e.g., compound of Formula I) or a pharmaceutically acceptable salt thereof, and a compound of Formula III, or a pharmaceutically acceptable salt thereof. Each of these different aspects can be described more particularly by the various embodiments described herein, which embodiments can be equally applicable to the different aspects.

Examples of the PDE10A inhibitor include, but are not limited to, papaverine, PF-02545920 (aka MP-10), RO5545965, TAK-063, AMG 579, and THPP-1. The structures of these PDE10A inhibitors are shown below:

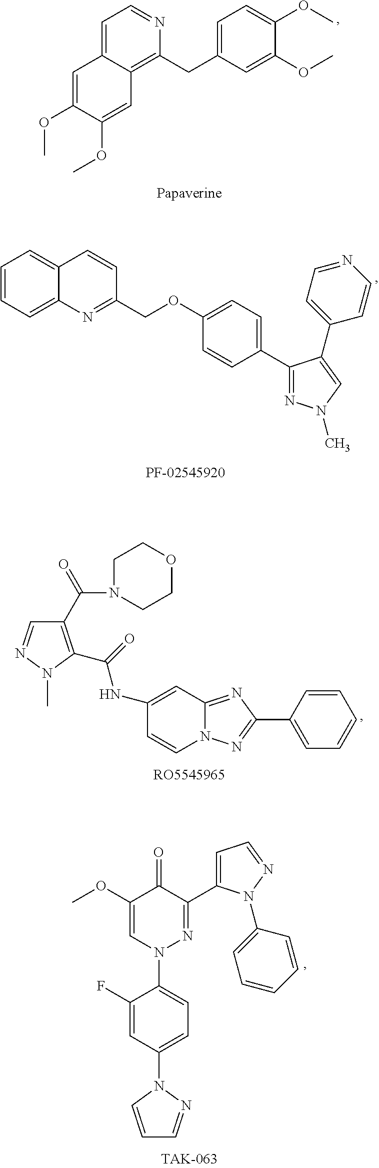

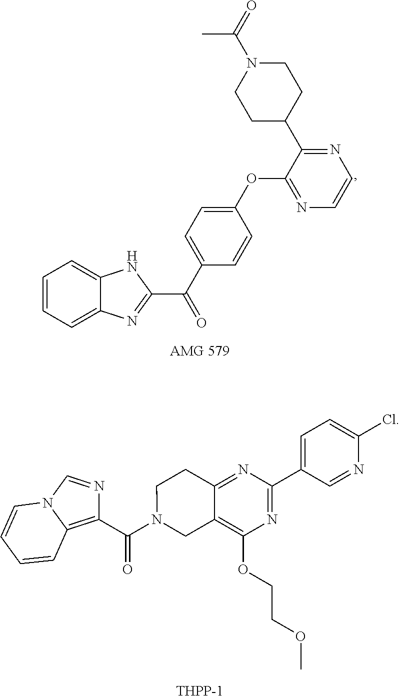 [SMILES: CC(=O)N1CCC(c2nccnc2Oc2ccc(C(=O)c3nc4ccccc4[nH]3)cc2)CC1 ; COCCOc1nc(-c2ccc(Cl)nc2)nc2c1CN(C(=O)c1ncn3ccccc13)CC2 ; "THPP-1"]

In certain embodiments, the method comprises administering the PDE10A inhibitor, or a pharmaceutically acceptable salt thereof, once daily. In certain embodiments, the method comprises administering orally the PDE10A inhibitor, or a pharmaceutically acceptable salt thereof. In certain embodiments, the method comprises administering the PDE10A inhibitor, or a pharmaceutically acceptable salt thereof, as a unit dose.

In another aspect, provided herein is a method of treating COFD, comprising administering to a subject in need thereof a composition containing a therapeutically effective amount of a therapeutic agent or a pharmaceutically acceptable salt thereof, wherein the therapeutic agent is a compound of Formula I (also referred to herein as RO5545965):

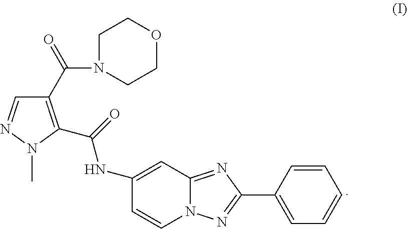 [SMILES: Cn1ncc(C(=O)N2CCOCC2)c1C(=O)Nc1ccn2nc(-c3ccccc3)nc2c1]

In a further aspect, provided herein is a method of treating COFD, comprising administering to a subject in need thereof a composition containing a therapeutically effective amount of a therapeutic agent or a pharmaceutically acceptable salt thereof, wherein the therapeutic agent is a compound of Formula I (i.e., RO5545965), wherein the compound in free base, or a pharmaceutically acceptable salt thereof, is administered in an amount of about 5 mg to about 15 mg once daily. In certain embodiments, the therapeutic agent is a compound of Formula I (i.e., RO5545965) in free base, or a pharmaceutically acceptable salt thereof, is administered in an amount of about 2.5 mg to about 15 mg once daily. In certain embodiments, the therapeutic agent is a compound of Formula I (i.e., RO5545965) in free base, or a pharmaceutically acceptable salt thereof, is administered in an amount of about 2.5 mg, about 5.0 mg, about 10 mg, or about 15 mg once daily.

Still another aspect of this invention is a method of treating COFD, comprising administering to a subject in need thereof a composition containing a therapeutically effective amount of a crystalline solid of the compound of Formula I above, wherein said crystalline solid has a melting point onset as determined by differential scanning calorimetry (DSC) of about 210° C. to about 214° C., and said administering comprises administering to the subject the crystalline solid in an amount of about 5 mg to about 15 mg once daily. In certain embodiments, a therapeutically effective amount of a crystalline solid of the compound of Formula I is administered in an amount of about 2.5 mg to about 15 mg once daily. In certain embodiments, a therapeutically effective amount of a crystalline solid of the compound of Formula I is administered in an amount of about 2.5 mg, about 5.0 mg, about 10 mg, or about 15 mg once daily.

In some embodiments, the crystalline solid set forth above has an XRPD pattern as substantially as substantially shown in FIG. 1. In some embodiments, the crystalline solid set forth above has a DSC curve as substantially shown in FIG. 2.

Also provided herein is a method of treating Childhood-Onset Fluency Disorder (COFD) in a subject in need thereof, comprising administering to a subject in need thereof a therapeutically effective amount of:

a compound of Formula III:

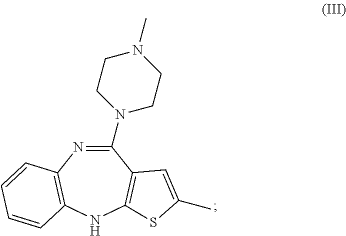 [SMILES: Cc1cc2c(s1)Nc1ccccc1N=C2N1CCN(C)CC1]

and
a compound of Formula I:

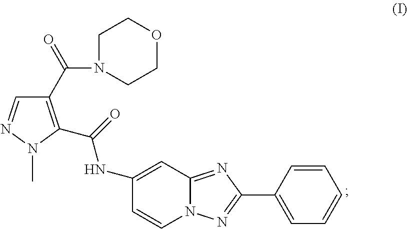 [SMILES: Cn1ncc(C(=O)N2CCOCC2)c1C(=O)Nc1ccn2nc(-c3ccccc3)nc2c1]

or a pharmaceutically acceptable salt thereof.

In certain embodiments, the compound of Formula III, or a pharmaceutically acceptable salt thereof, is administered at about 2.5 mg to about 5.0 mg once daily; and the compound of Formula I, or a pharmaceutically acceptable salt thereof, is administered at about 5.0 mg to about 15 mg once daily.

Also provided herein is a compound of the compound of Formula III:

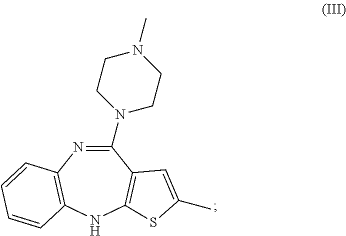 [SMILES: Cc1cc2c(s1)Nc1ccccc1N=C2N1CCN(C)CC1]

and
a crystalline solid of Formula I.

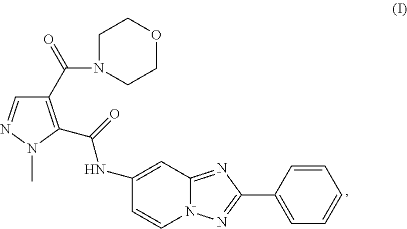 [SMILES: Cn1ncc(C(=O)N2CCOCC2)c1C(=O)Nc1ccn2nc(-c3ccccc3)nc2c1]

wherein said crystalline solid has a melting point onset as determined by DSC of about 210° C. to about 214° C.; or a pharmaceutically acceptable salt thereof. In certain embodiments, the compound of Formula III, or a pharmaceutically acceptable salt thereof, is administered at about 2.5 mg to about 5.0 mg once daily; and the crystalline solid of Formula I, or a pharmaceutically acceptable salt thereof, is administered at about 5.0 mg to about 15 mg once daily.

The details of one or more embodiments of the invention are set forth in the description below. Other features, objects, and advantages of the invention will be apparent from the drawings, description and from the claims.

### BRIEF DESCRIPTION OF THE DRAWINGS

FIG. 1 depicts an exemplary XRPD pattern of a crystalline solid of the free base of the compound of Formula I.

FIG. 2 depicts an exemplary DSC curve of a crystalline solid of the free base of the compound of Formula I.

FIG. 3 depicts the glucose level during oral glucose tolerance test in Sprague Dawley rats after acute treatment with Olanzapine (compound of Formula III) or Haloperidol.

FIG. 4 depicts the insulin level of Sprague Dawley rats after oral glucose challenge (2 g/kg).

FIG. 5 depicts the oral glucose tolerance test and glucose level in Sprague Dawley rats after acute treatment with Olanzapine (compound of Formula III) or Haloperidol.

FIG. 6 depicts the body weight and food intake of Sprague Dawley rats during treatment of Olanzapine (compound of Formula III) or RO5545965 (compound of Formula I).

FIG. 7 depicts the oral glucose tolerance test and glucose level in Sprague Dawley rats after sub-chronic treatment with Olanzapine (compound of Formula III), RO5545965 (compound of Formula I), or a combination of Olanzapine (compound of Formula III) and the compound of Formula I.

FIG. 8 depicts the insulin level in Sprague Dawley rats before the oral glucose tolerance test with Olanzapine (compound of Formula III), RO5545965 (compound of Formula I), and a combination of Olanzapine (compound of Formula III) and the compound of Formula I.

### DETAILED DESCRIPTION

As generally described herein, the present disclosure provides methods of treating COFD in a subject in need thereof. The present disclosure also describes use of the specific PDE10A inhibitor RO5545965 for treating COFD.

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from the group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred from the context. For example, the term “about 10 mg” means 10 mg with a ±10% variation from 10 mg, i.e., an amount in the range of 9 mg to 11 mg.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

As used herein, “pharmaceutical composition” or “pharmaceutical formulation” refers to the combination of an active agent with an excipient or a carrier, inert or active, making the composition especially suitable for diagnostic or therapeutic use in vivo or ex vivo.

“Pharmaceutically acceptable” means approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable salt” refers to any salt of an acidic or a basic group that may be present in a compound of the present invention (e.g., the compound of formula (I)), which salt is compatible with pharmaceutical administration.

As is known to those of skill in the art, “salts” of compounds may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds described herein and their pharmaceutically acceptable acid addition salts.

Examples of bases include, but are not limited to, alkali metal (e.g., sodium and potassium) hydroxides, alkaline earth metal (e.g., magnesium and calcium) hydroxides, ammonia, and compounds of formula NW₄⁺, wherein W is C₁₋₄ alkyl, and the like.

Examples of salts include, but are not limited, to acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, K⁺, Ca²⁺, NH₄⁺, and NW₄⁺ (where W can be a C₁₋₄ alkyl group), and the like.

For therapeutic use, salts of the compounds of the present disclosure are contemplated as being pharmaceutically acceptable. However, salts of acids and bases that are non-pharmaceutically acceptable may also find use, for example, in the preparation or purification of a pharmaceutically acceptable compound.

As used herein, “pharmaceutically acceptable excipient” refers to a substance that aids the administration of an active agent to and/or absorption by a subject and can be included in the compositions of the present invention without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. For examples of excipients, see Martin, Remington's Pharmaceutical Sciences, 15th Ed., Mack Publ. Co., Easton, PA (1975).

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

As used herein, “solid dosage form” means a pharmaceutical dose(s) in solid form, e.g., tablets, capsules, granules, powders, sachets, reconstitutable powders, dry powder inhalers and chewables.

As used herein, “administering” means oral administration, administration as a suppository, topical contact, intravenous administration, parenteral administration, intraperitoneal administration, intramuscular administration, intralesional administration, intrathecal administration, intracranial administration, intranasal administration or subcutaneous administration, transmucosal (e.g., buccal, sublingual, nasal, or transdermal) administration, or the implantation of a slow-release device, e.g., a mini-osmotic pump, to a subject. Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc.

By “co-administer” it is meant that a composition described herein is administered at the same time, just prior to, or just after the administration of one or more additional therapies (e.g., a dopamine receptor antagonist, an antipsychotic, or treatment for a neuro-developmental disease). The PDE10A inhibitor described above, or a pharmaceutically acceptable salt thereof, can be administered alone or can be co-administered to a subject. Co-administration is meant to include simultaneous or sequential administration of the compound individually or in combination (more than one compound or agent). Thus, the preparations can also be combined, when desired, with other active substances (e.g., to reduce dopamine hyperactivity).

As used herein, the term“administered simultaneously” as used herein is not specifically restricted and means that the components of the combination therapy are substantially administered at the same time, e.g. as a mixture or in immediate subsequent sequence. The term“administered successively” as used herein is not specifically restricted and means that the components of the combination therapy are not administered at the same time but one after the other, or in groups, with a specific time interval between.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (e.g., “therapeutic treatment”). Subjects are effectively treated whenever a clinically beneficial result ensues. This may mean, for example, a complete or marked resolution of the symptoms of a disorder, a decrease in the frequency, severity, and/or duration of the symptoms, or a slowing of the disorder's progression. Thus, an effective treatment could manifest as a decrease in the number, duration, frequency and/or intensity of repetitions, prolongations, hesitations and interruptions in the flow of speech observed in the subject.

The phrase “therapeutically effective amount” as used herein means an amount of a composition (e.g., a composition described herein), or a compound of Formula I, or a pharmaceutically acceptable salt thereof, which is effective for producing some desired therapeutic effect in a subject.

Childhood-onset fluency disorder (COFD), also referred to as stuttering, stammering, or dysphemia, is a communication disorder characterized by involuntary repetitions and prolongations of sounds, syllables, words, or phrases as well as involuntary silent pauses or blocks in which the person who stutters is unable to produce sounds. In certain embodiments, administration of a composition (e.g., a composition described herein) improves COFD symptoms.

In certain embodiments, one or more compounds disclosed herein are useful in the treatment of speech and language disorders including expressive language disorder, mixed receptive-expressive language disorder, phonological disorder, and communication disorder not-otherwise-specified (DSM-IV).

The term “stuttering” covers a wide range of severity, from barely perceptible impediments that are largely cosmetic to severe symptoms that effectively prevent oral communication. Almost 70 million people worldwide stutter, among which four-fifths of stutterers are male. It is common for individuals who suffer from a lifetime of stuttering for their symptoms to worsen considerably as they reach their 70s and 80s.

The impact of COFD on a person's functioning and emotional state can be severe. This may include fears of having to enunciate specific vowels or consonants, fears of being caught stuttering in social situations, self-imposed isolation, anxiety, stress, shame, low self-esteem, being a possible target of bullying (especially in children), having to use word substitution and rearrange words in a sentence to hide stuttering, or a feeling of “loss of control” during speech. Symptoms of COFD develop between the ages of 2 and 7, with 80 to 90 percent of cases developing by age 6. While mild stuttering is common in children who are learning to speak, this behavior becomes a fluency disorder when it persists over time and causes distress in the child.

### Phosphodiesterase Inhibitors

Phosphodiesterases are a diverse family of enzymes that hydrolyse cyclic nucleotides and thus play a key role in regulating intracellular levels of the second messengers cyclic adenosine monophosphate (cAMP) and cyclic guanosine monophosphate (cGMP). In certain embodiments, compounds of the present disclosure block the enzyme phosphodiesterase, thereby preventing the activation of one or more intracellular second messengers.

### Compound

As set forth in the SUMMARY section above, the PDE10A inhibitor that may be used in the methods of this invention can be one of the following compounds, or a pharmaceutically acceptable salt thereof:

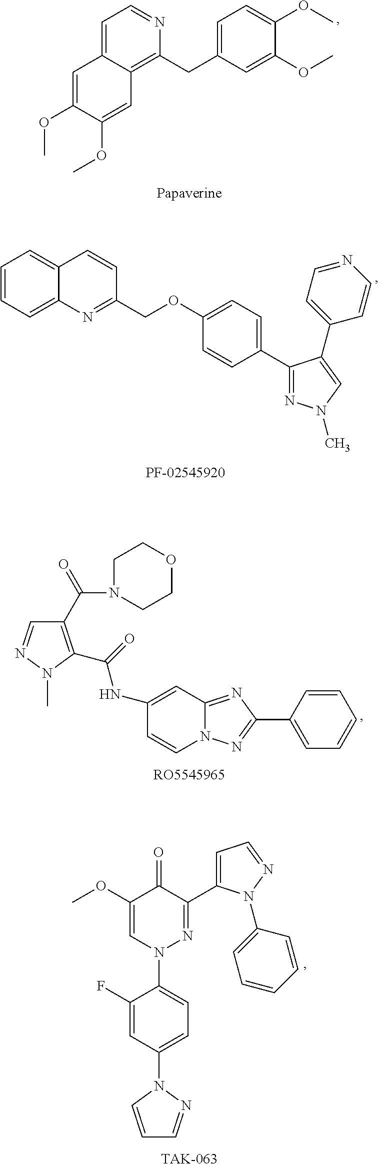

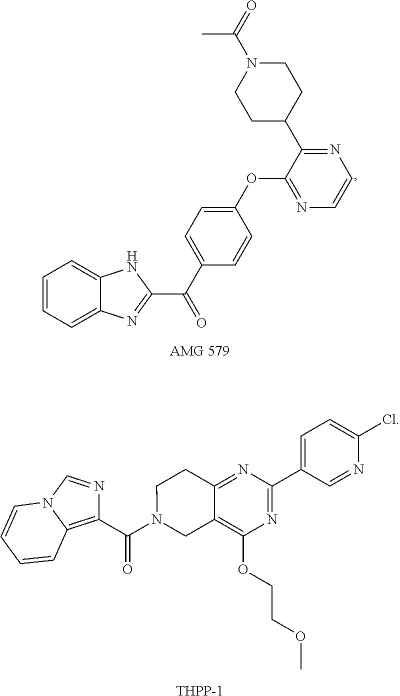 [SMILES: CC(=O)N1CCC(c2nccnc2Oc2ccc(C(=O)c3nc4ccccc4[nH]3)cc2)CC1 ; COCCOc1nc(-c2ccc(Cl)nc2)nc2c1CN(C(=O)c1ncn3ccccc13)CC2 ; "THPP-1"]

Syntheses of the above listed PDE10A inhibitors can follow procedures known in the field. For example, a method of chemically synthesizing the compound of RO5545965 (including Example 1 provided herein, infra) is described in U.S. Pat. No. 8,349,824, which is incorporated by reference in its entirety.

The compound of Formula I, as depicted below, is a phosphodiesterase 10A (PDE10A) inhibitor, also known as RO5545965 with the chemical name of 1-methyl-4-(morpholine-4-carbonyl)-N-(2-phenyl[1,2,4]triazolo[1,5-a]pyridin-7-yl)-1H-pyrazole-5-carboxamide.

### Structure of Compound of Formula 1:

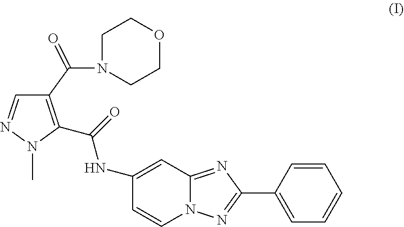 [SMILES: Cn1ncc(C(=O)N2CCOCC2)c1C(=O)Nc1ccn2nc(-c3ccccc3)nc2c1]

The term “compound of Formula I” may also be referenced as “Compound 1” or “RO554965.”

In various embodiments, the pharmaceutically acceptable salt of the compound of Formula I can be a salt of the compound of formula (I) with physiologically compatible mineral acids, such as hydrochloric acid, sulphuric acid, sulphurous acid or phosphoric acid; or with organic acids, such as methanesulphonic acid, p-toluenesulphonic acid, acetic acid, lactic acid, trifluoroacetic acid, citric acid, fumaric acid, maleic acid, tartaric acid, succinic acid or salicylic acid.

The compound of Formula III, as depicted below, is also known as Olanzapine or ZYPREXA with a chemical name of 10H-Thieno[2,3-b][1,5]benzodiazepine, 2-methyl-4-(4-methyl-1-piperazinyl)- or 2-Methyl-4-(4-methyl-1-piperazinyl)-10H-thieno[2,3-b][1,5]benzodiazepine and a CAS number of 132539-06-1.

### Structure of Compound of Formula III:

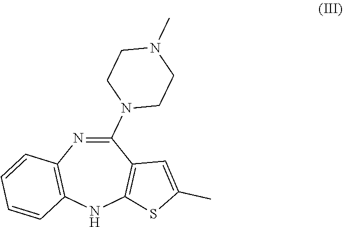 [SMILES: Cc1cc2c(s1)Nc1ccccc1N=C2N1CCN(C)CC1]

The term “compound of Formula III” may also be referenced as “Compound 3” or “Olanzapine.”

In various embodiments, the pharmaceutically acceptable salt of the compound of Formula III can be a salt of the compound of Formula III with physiologically compatible mineral acids, such as hydrochloric acid, sulphuric acid, sulphurous acid or phosphoric acid; or with organic acids, such as methanesulphonic acid, p-toluenesulphonic acid, acetic acid, lactic acid, trifluoroacetic acid, citric acid, fumaric acid, maleic acid, tartaric acid, succinic acid or salicylic acid.

The present disclosure covers a method of treating COFD, comprising administering to a subject in need thereof a composition containing a therapeutically effective amount of a therapeutic agent or a pharmaceutically acceptable salt thereof, wherein the therapeutic agent is a compound of Formula I (aka RO5545965):

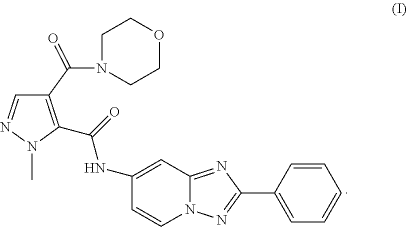 [SMILES: Cn1ncc(C(=O)N2CCOCC2)c1C(=O)Nc1ccn2nc(-c3ccccc3)nc2c1]

In certain embodiments, the compound is administered in an amount of about 1 mg to about 17 mg once daily. In certain embodiments, the compound is administered in an amount of about 2.5 mg to about 15 mg once daily. In some embodiments, the compound is administered in an amount of about 5 mg to about 15 mg once daily. In certain embodiments, the therapeutic agent is a compound of Formula I (i.e., RO5545965) in free base, or a pharmaceutically acceptable salt thereof, is administered in an amount of about 2.0 mg, about 2.5 mg, about 5.0 mg, about 10 mg, or about 15 mg once daily.

The present disclosure also covers a method of treating COFD, comprising administering to a subject in need thereof a composition containing a therapeutically effective amount of a crystalline solid of the compound of Formula I:

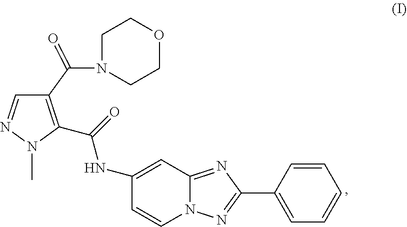 [SMILES: Cn1ncc(C(=O)N2CCOCC2)c1C(=O)Nc1ccn2nc(-c3ccccc3)nc2c1]

wherein said crystalline solid has a melting point onset as determined by differential scanning calorimetry (DSC) of about 210° C. to about 214° C. (e.g., about 210° C., about 211° C., about 212° C., about 213° C., or about 214° C.), and said administering comprises administering the crystalline solid in an amount of about 5 mg to about 15 mg once daily.

The present disclosure also covers a method of treating COFD, comprising administering to a subject in need thereof a composition containing a therapeutically effective amount of a crystalline solid of the compound of Formula I:

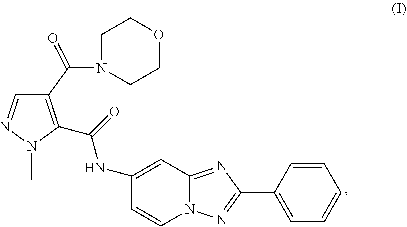 [SMILES: Cn1ncc(C(=O)N2CCOCC2)c1C(=O)Nc1ccn2nc(-c3ccccc3)nc2c1]

wherein said crystalline solid has a melting point onset as determined by differential scanning calorimetry (DSC) of about 210° C. to about 214° C. (e.g., about 210° C., about 211° C., about 212° C., about 213° C., or about 214° C.), and said administering comprises administering the crystalline solid in an amount of about 2.5 mg to about 15 mg once daily.

Also provided herein is a method of treating Childhood-Onset Fluency Disorder (COFD) in a subject in need thereof, comprising administering to a subject in need thereof a therapeutically effective amount of: a compound of Formula III:

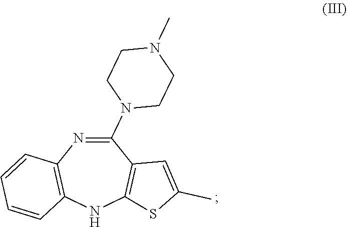 [SMILES: Cc1cc2c(s1)Nc1ccccc1N=C2N1CCN(C)CC1]

and
a compound of Formula I:

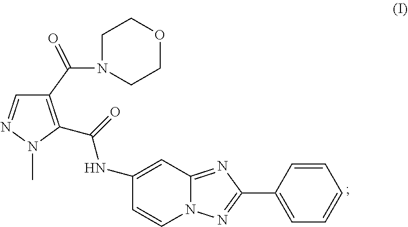 [SMILES: Cn1ncc(C(=O)N2CCOCC2)c1C(=O)Nc1ccn2nc(-c3ccccc3)nc2c1]

or a pharmaceutically acceptable salt thereof.

In certain embodiments, the compound of Formula III, or a pharmaceutically acceptable salt thereof, is administered at about 2.5 mg to about 5.0 mg once daily; and the compound of Formula I, or a pharmaceutically acceptable salt thereof, is administered at about 5.0 mg to about 15 mg once daily.

Also provided herein is a compound of the compound of Formula III:

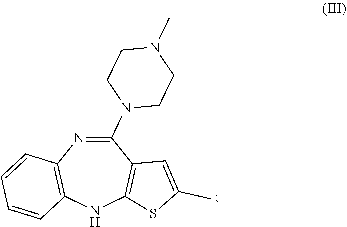 [SMILES: Cc1cc2c(s1)Nc1ccccc1N=C2N1CCN(C)CC1]

and
a crystalline solid of Formula I:

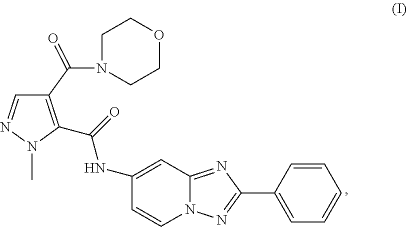 [SMILES: Cn1ncc(C(=O)N2CCOCC2)c1C(=O)Nc1ccn2nc(-c3ccccc3)nc2c1]

wherein said crystalline solid has a melting point onset as determined by DSC of about 210° C. to about 214° C.; or a pharmaceutically acceptable salt thereof.

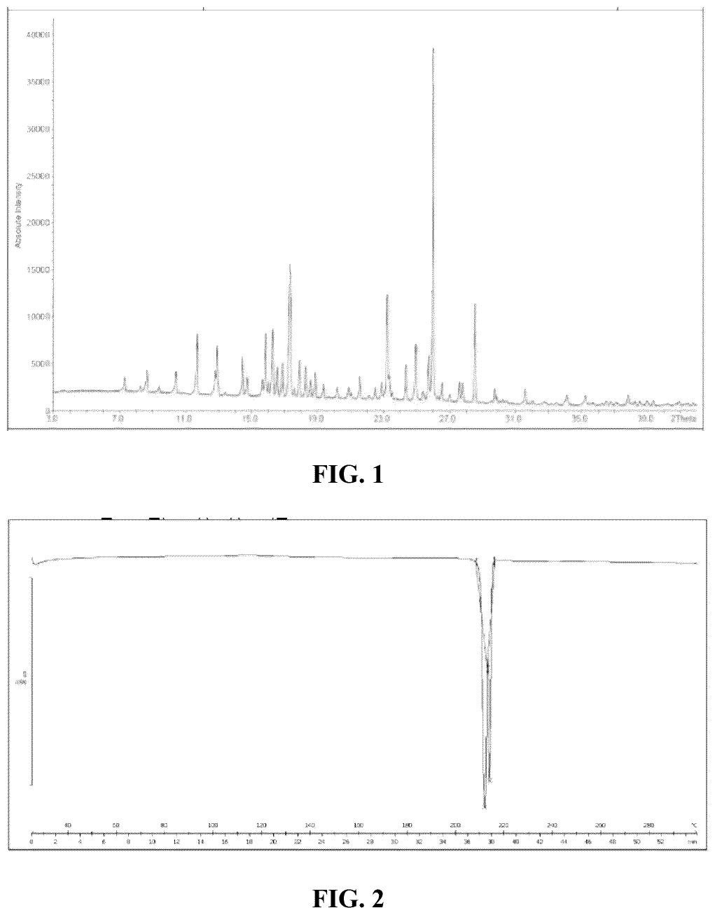

In some embodiments, the above described crystalline solid of the free base of Formula I has an X-ray powder diffraction (XRPD) pattern as substantially shown in FIG. 1.

In some embodiments, the above described crystalline solid of the free base of Formula I has a DSC figure as substantially shown in FIG. 2.

### Pharmaceutical Compositions

In one aspect, provided herein is a method of treating COFD using a pharmaceutical composition comprising a PDE10A inhibitor (e.g., the compound of Formula I or RO5545965), or a pharmaceutically acceptable salt thereof, and a pharmaceutically acceptable excipient, for the treatment of COFD in a subject in need thereof.

Typically, the PDE10A inhibitor used in the method of this invention has no effect on insulin resistance.

In various embodiments, the method administers a composition comprising the PDE10A inhibitor, or a pharmaceutically acceptable salt thereof, as the sole active agent.

In various embodiments, the method administers a composition comprising the PDE10A inhibitor, or a pharmaceutically acceptable salt thereof, in combination with another therapeutically active agent.

In some embodiments, the other therapeutically active agent is a dopamine receptor antagonist.

In some embodiments, the other therapeutically active agent is a dopamine receptor D1 (DRD1) antagonist. An exemplary DRD1 antagonist is ecopipam.

In some embodiments, the other therapeutically active agent is a dopamine receptor D2 (DRD2) antagonist. An exemplary DRD2 antagonist is Olanzapine, Risperidone, Lurasidone, or Pimozide.

In various embodiments, the amount of the PDE10A inhibitor (e.g., the compound of Formula I, also known as RO5545965), or a pharmaceutically acceptable salt thereof, in the pharmaceutical compositions described herein can be from about 1 mg to about 100 mg, about 2 mg to about 50 mg, about 3 mg to about 20 mg, about 5 mg to about 15 mg, about 5 mg to about 10 mg or about 2.5 mg to about 15 mg.

In certain embodiments, the amount of the PDE10A inhibitor (e.g., the compound of Formula I or RO5545965), or a pharmaceutically acceptable salt thereof, in the pharmaceutical compositions described herein can be from about 1.0 mg to about 17 mg, about 2.0 mg to about 16 mg, about 2.5 mg to about 15 mg, about 5.5 mg to about 15 mg, about 6 mg to about 15 mg, about 6.5 mg to about 15 mg, about 7 mg to about 15 mg, about 7.5 mg to about 15 mg, about 8 mg to about 15 mg, about 8.5 mg to about 15 mg, about 9 mg to about 15 mg, about 9.5 mg to about 15 mg, about 10 mg to about 15 mg, about 10.5 mg to about 15 mg, about 11 mg to about 15 mg, about 11.5 mg to about 15 mg, about 12 mg to about 15 mg, about 12.5 mg to about 15 mg, about 13 mg to about 15 mg, about 13.5 mg to about 15 mg, or about 14 mg to about 15 mg.

In certain embodiments, the amount of the PDE10A inhibitor (e.g., the compound of Formula I or RO5545965), or a pharmaceutically acceptable salt thereof, in the pharmaceutical compositions described herein can be from about 5 mg to about 14.5 mg, about 5 mg to about 14 mg, about 5 mg to about 13.5 mg, about 5 mg to about 13 mg, about 5 mg to about 12.5 mg, about 5 mg to about 12 mg, about 5 mg to about 11.5 mg, about 5 mg to about 11 mg, about 5 mg to about 10.5 mg, about 5 mg to about 10 mg, about 5 mg to about 9.5 mg, about 5 mg to about 9 mg, about 5 mg to about 8.5 mg, about 5 mg to about 8 mg, about 5 mg to about 7.5 mg, about 5 mg to about 7 mg, about 5 mg to about 6.5 mg, or about 5 mg to about 6 mg.

In certain embodiments, the amount of the PDE10A inhibitor (e.g., the compound of Formula I or RO5545965), or a pharmaceutically acceptable salt thereof, in the pharmaceutical compositions described herein can be from about 5 mg to about 15 mg (e.g., about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, or about 15 mg).

In some embodiments, the PDE10A inhibitor, or a pharmaceutically acceptable salt thereof, is administered at about 1 mg to about 100 mg once daily.

In some embodiments, the PDE10A inhibitor, or a pharmaceutically acceptable salt thereof, is administered at about 5 mg to about 15 mg once daily.

In some embodiments, the other therapeutically active agent is administered at about 0.1 mg to about 10 mg once daily.

In some embodiments, the other therapeutically active agent is administered at about 0.5 mg to about 5 mg once daily.

In some embodiments, the PDE10A inhibitor, or a pharmaceutically acceptable salt thereof, is administered at about 5 mg to about 15 mg once daily; and the dopamine receptor antagonist is administered at about 0.5 mg to about 5 mg once daily.

In certain embodiments, the PDE10A inhibitor, or a pharmaceutically acceptable salt thereof, is administered at about 5 mg to about 15 mg once daily; and the compound of Formula III is administered at about 2.5 mg to about 5.0 mg once daily. In certain embodiments, the compound of Formula I, or a pharmaceutically acceptable salt thereof, is administered at about 5 mg to about 15 mg once daily; and the compound of Formula III is administered at about 2.5 mg to about 5.0 mg once daily.

In embodiments, the compound of Formula III, or a pharmaceutically acceptable salt thereof, in the pharmaceutical compositions described herein can be from about 2.5 mg to about 5.0 mg (e.g., about 2.5 mg, about 2.6 mg, about 2.7 mg, about 2.8 mg, about 2.9 mg, about 3.0 mg, about 3.1 mg, about 3.2 mg, about 3.3 mg, about 3.4 mg, about 3.5 mg, about 3.6 mg, about 3.7 mg, about 3.8 mg, about 3.9 mg, about 4.0 mg, about 4.1 mg, about 4.2 mg, about 4.3 mg, about 4.4 mg, about 4.5 mg, about 4.6 mg, about 4.7 mg, about 4.8 mg, about 4.9 mg, about 5.0 mg).

In various embodiments, the amount of the compound of Formula I, or a pharmaceutically acceptable salt thereof, in the pharmaceutical compositions described herein can be from about 1.0 mg to about 17 mg. In various embodiments, the amount of the compound of Formula I, or a pharmaceutically acceptable salt thereof, in the pharmaceutical compositions described herein can be from about 2.5 mg to about 15 mg. In certain embodiments, the amount of the compound of Formula I, or a pharmaceutically acceptable salt thereof, in the pharmaceutical compositions described herein can be about 2.5 mg. In certain embodiments, the amount of the compound of Formula I, or a pharmaceutically acceptable salt thereof, in the pharmaceutical compositions described herein can be about 5.0 mg. In certain embodiments, the amount of the compound of Formula I, or a pharmaceutically acceptable salt thereof, in the pharmaceutical compositions described herein can be about 10 mg. In certain embodiments, the amount of the compound of Formula I, or a pharmaceutically acceptable salt thereof, in the pharmaceutical compositions described herein can be about 15 mg.

In certain embodiments, the compound of Formula I, or a pharmaceutically acceptable salt thereof, is administered at about 2.5 mg to about 15 mg once daily.

In certain embodiments, the compound of Formula I, or a pharmaceutically acceptable salt thereof, is administered at about 2.5 mg once daily.

In certain embodiments, the compound of Formula I, or a pharmaceutically acceptable salt thereof, is administered at about 5.0 mg once daily.

In certain embodiments, the compound of Formula I, or a pharmaceutically acceptable salt thereof, is administered at about 10 mg once daily.

In certain embodiments, the compound of Formula I, or a pharmaceutically acceptable salt thereof, is administered at about 15 mg once daily.

In various embodiments, the pharmaceutical compositions described herein comprise a therapeutically effective amount of the free base form of the compound of Formula I.

In various embodiments, the pharmaceutical compositions described herein comprise a therapeutically effective amount of a pharmaceutically acceptable salt of the compound of Formula I. In some embodiments, the pharmaceutically acceptable salt of the compound of Formula I can be a salt of the compound of formula (I) with physiologically compatible mineral acids, such as hydrochloric acid, sulphuric acid, sulphurous acid or phosphoric acid; or with organic acids, such as methanesulphonic acid, p-toluenesulphonic acid, acetic acid, lactic acid, trifluoroacetic acid, citric acid, fumaric acid, maleic acid, tartaric acid, succinic acid or salicylic acid.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration. In certain embodiments, the pharmaceutical compositions disclosed herein are administered orally.

The pharmaceutical compositions provided herein may also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc., or may be continued indefinitely, for example, for the rest of the subject's life. In certain embodiments, the chronic administration is intended to provide a constant level of the compound in the blood, e.g., within the therapeutic window over the extended period of time.

The pharmaceutical compositions provided herein may be presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. In various embodiments, the pharmaceutical dosage forms described herein can be administered as a unit dose. Typical unit dosage forms include prefilled, premeasured ampules or syringes of the liquid compositions or pills, tablets, capsules or the like in the case of solid compositions.

In various embodiments, the pharmaceutical compositions provided herein are administered to the patient as a solid dosage form. In certain embodiments, the solid dosage form is a capsule. In certain embodiments, the solid dosage form is a tablet.

In various embodiments, the pharmaceutical compositions provided herein comprise the compound of Formula I as the sole active agent, or in combination with other active agents.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st} ed., Lippincott Williams & Wilkins, 2005.

### Methods of Use and Treatment

In one aspect, provided herein are methods for treating COFD in a subject in need thereof, comprising administering to a subject in need thereof a composition containing a therapeutically effective amount of a therapeutic agent or a pharmaceutically acceptable salt thereof, wherein the therapeutic agent is a compound of Formula I.

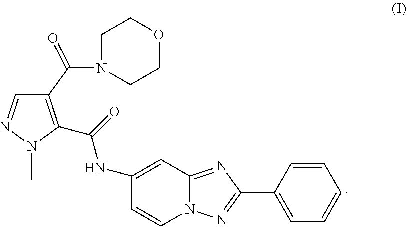 [SMILES: Cn1ncc(C(=O)N2CCOCC2)c1C(=O)Nc1ccn2nc(-c3ccccc3)nc2c1]

In various embodiments, administering a therapeutically effective amount of the compound of Formula I, or a pharmaceutically acceptable salt thereof, comprises administering a composition having an amount of the compound as described herein, supra.

In various embodiments, the composition comprises the compound of Formula I, or a pharmaceutically acceptable salt thereof, as the sole active agent.

In various embodiments, the composition comprises the compound of Formula I, or a pharmaceutically acceptable salt thereof, in combination with another active agent. In certain embodiments, the other active agent is a dopamine receptor antagonist (e.g., a D1 antagonist or a D2 antagonist).

In various embodiments, the composition comprises inactive agents selected from the group consisting of mannitol, microcrystalline cellulose, sodium starch glycolate, sucrose monopalmitate, hydroxypropyl methylcellulose, colloidal silicon dioxide, and sodium stearyl fumarate.

In various embodiments, administering comprises administering the compound of Formula I, or a pharmaceutically acceptable salt thereof, in a capsule.

In various embodiments, the capsule shell consists of gelatine, titanium dioxide, red iron oxide, and yellow iron oxide.

In various embodiments, the capsule described above comprises the compound of Formula I (or RO5545965), or a pharmaceutically acceptable salt thereof, in the amount from about 1 mg to about 100 mg, about 2 mg to about 50 mg, about 3 mg to about 20 mg, or about 5 mg to about 15 mg. In certain embodiments, the capsule described above comprises the compound of Formula I (or RO5545965), or a pharmaceutically acceptable salt thereof, in the amount from about 5.5 mg to about 10 mg, about 6 mg to about 10 mg, about 6.5 mg to about 10 mg, about 7 mg to about 10 mg, about 7.5 mg to about 10 mg, about 8 mg to about 10 mg, about 8.5 mg to about 10 mg, about 9 mg to about 10 mg, about 5 mg to about 9.5 mg, about 5 mg to about 9 mg, about 5 mg to about 8.5 mg, about 5 mg to about 8 mg, about 5 mg to about 7.5 mg, about 5 mg to about 7 mg, about 5 mg to about 6.5 mg, or about 5 mg to about 6 mg.

In certain embodiments, the capsule described above comprises the compound of Formula I (or RO5545965), or a pharmaceutically acceptable salt thereof, in the amount from about 5 mg to about 15 mg (e.g., about 5 mg, about 5.5 mg, about 6 mg, about 6.5 mg, about 7 mg, about 7.5 mg, about 8 mg, about 8.5 mg, about 9 mg, about 9.5 mg, or about 10 mg).

In various embodiments, the capsule described above comprises the compound of Formula I (or RO5545965), or a pharmaceutically acceptable salt thereof, in the amount from about 1.0 mg to about 17 mg. In various embodiments, the capsule described above comprises the compound of Formula I (or RO5545965), or a pharmaceutically acceptable salt thereof, in the amount from about 2.5 mg to about 15 mg. In certain embodiments, the capsule described above comprises the compound of Formula I (or RO5545965), or a pharmaceutically acceptable salt thereof, in the amount of about 2.5 mg. In certain embodiments, the capsule described above comprises the compound of Formula I (or RO5545965), or a pharmaceutically acceptable salt thereof, in the amount of about 5.0 mg. In certain embodiments, the capsule described above comprises the compound of Formula I (or RO5545965), or a pharmaceutically acceptable salt thereof, in the amount of about 10 mg.

In some embodiments, administering comprises administering the compound of Formula I (or RO5545965), or a pharmaceutically acceptable salt thereof, in an amount of about 5 mg to about 15 mg once daily.

In some embodiments, administering comprises administering the compound of Formula I (or RO5545965), or a pharmaceutically acceptable salt thereof, in an amount of about 2.5 mg to about 15 mg once daily. In some embodiments, administering comprises administering the compound of Formula I (or RO5545965), or a pharmaceutically acceptable salt thereof, in an amount of about 2.5 mg once daily. In some embodiments, administering comprises administering the compound of Formula I (or RO5545965), or a pharmaceutically acceptable salt thereof, in an amount of about 5.0 mg once daily. In some embodiments, administering comprises administering the compound of Formula I (or RO5545965), or a pharmaceutically acceptable salt thereof, in an amount of about 10 mg once daily. In some embodiments, administering comprises administering the compound of Formula I (or RO5545965), or a pharmaceutically acceptable salt thereof, in an amount of about 15 mg once daily.

In various embodiments, administering comprises administering the compound of Formula I, or a pharmaceutically acceptable salt thereof, in an immediate release formulation.

In various embodiments, administering comprises administering the compound of Formula I, or a pharmaceutically acceptable salt thereof, in an extended release formulation.

In various embodiments, administering maintains efficacy throughout the day.

In various embodiments, the compound of Formula I, or a pharmaceutically acceptable salt thereof, is administered once, twice, three, four, or five times daily. In certain embodiments, the compound of Formula I, or a pharmaceutically acceptable salt thereof, is administered once daily. In certain embodiments, the compound of Formula I, or a pharmaceutically acceptable salt thereof, is administered twice daily.

In various embodiments, the compound of Formula I, or a pharmaceutically acceptable salt thereof, is administered orally.

In various embodiments, the compound of Formula I, or a pharmaceutically acceptable salt thereof, is administered as a unit dose.

In various embodiments, the compound of Formula I is administered in free base form.

In various embodiments, the compound of Formula I is administered in the form of a pharmaceutically acceptable salt. In certain embodiments, the pharmaceutically acceptable salt of the compound of Formula I can be a salt of the compound of formula (I) with physiologically compatible mineral acids, such as hydrochloric acid, sulphuric acid, sulphurous acid or phosphoric acid; or with organic acids, such as methanesulphonic acid, p-toluenesulphonic acid, acetic acid, lactic acid, trifluoroacetic acid, citric acid, fumaric acid, maleic acid, tartaric acid, succinic acid or salicylic acid. In certain embodiments, the compound of Formula I is administered in a crystalline form.

Also provided herein is a method of treating Childhood-Onset Fluency Disorder (COFD) in a subject in need thereof, comprising administering to a subject in need thereof a therapeutically effective amount of: a compound of Formula III:

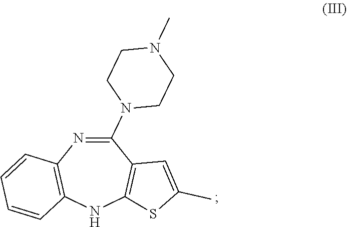 [SMILES: Cc1cc2c(s1)Nc1ccccc1N=C2N1CCN(C)CC1]

and
a compound of Formula I:

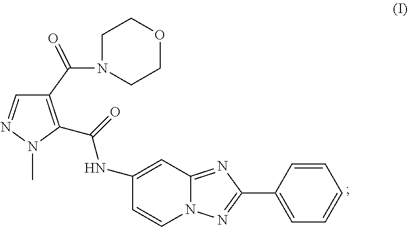 [SMILES: Cn1ncc(C(=O)N2CCOCC2)c1C(=O)Nc1ccn2nc(-c3ccccc3)nc2c1]

or a pharmaceutically acceptable salt thereof.

In embodiments, the compound of Formula III, or a pharmaceutically acceptable salt thereof, is administered at about 1 mg to about 500 mg once daily; and the compound of Formula I, or a pharmaceutically acceptable salt thereof, is administered at about 1 mg to about 500 mg once daily.

In embodiments, the compound of Formula III, or a pharmaceutically acceptable salt thereof, is administered at about 1 mg to about 50 mg once daily; and the compound of Formula I, or a pharmaceutically acceptable salt thereof, is administered at about 1 mg to about 50 mg once daily.

In embodiments, the compound of Formula III, or a pharmaceutically acceptable salt thereof, is administered at about 1 mg to about 30 mg once daily; and the compound of Formula I, or a pharmaceutically acceptable salt thereof, is administered at about 500 μg to about 20 mg once daily.

In embodiments, the compound of Formula III, or a pharmaceutically acceptable salt thereof, is administered at about 0.1 mg to about 10 mg once daily; and the compound of Formula I, or a pharmaceutically acceptable salt thereof, is administered at about 1 mg to about 20 mg once daily.

In embodiments, the compound of Formula III, or a pharmaceutically acceptable salt thereof, is administered at about 2.5 mg to about 5.0 mg once daily; and the compound of Formula I, or a pharmaceutically acceptable salt thereof, is administered at about 5.0 mg to about 15 mg once daily.

In embodiments, the compound of Formula III, or a pharmaceutically acceptable salt thereof, is administered at about 2.5 mg to about 5.0 mg (e.g., about 2.5 mg, about 3.0 mg, about 3.5 mg, about 4.0 mg, about 4.5 mg, about 5.0 mg) once daily; and the compound of Formula I, or a pharmaceutically acceptable salt thereof, is administered at about 5.0 mg to about 15 mg (e.g., about 5.0 mg, about 6.0 mg, about 7.0 mg, about 8.0 mg, about 9.0 mg, about 10 mg, about 11 mg, about 12 mg, about 13 mg, about 14 mg, about 15 mg) once daily.

In embodiments, the compound of Formula III, or a pharmaceutically acceptable salt thereof, is administered at about 1 mg to about 6.0 mg once daily; and the compound of Formula I, or a pharmaceutically acceptable salt thereof, is administered at about 4.0 mg to about 16 mg once daily. In embodiments, the compound of Formula III, or a pharmaceutically acceptable salt thereof, is administered at about 2.5 mg, about 2.6 mg, about 2.7 mg, about 2.8 mg, about 2.9 mg, about 3.0 mg, about 3.1 mg, about 3.2 mg, about 3.3 mg, about 3.4 mg, about 3.5 mg, about 3.6 mg, about 3.7 mg, about 3.8 mg, about 3.9 mg, about 4.0 mg, about 4.1 mg, about 4.2 mg, about 4.3 mg, about 4.4 mg, about 4.5 mg, about 4.6 mg, about 4.7 mg, about 4.8 mg, about 4.9 mg, about 5.0 mg; and the compound of Formula I, or a pharmaceutically acceptable salt thereof, is administered at about 5.0 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, or about 15 mg.

In embodiments, the compound of Formula III, or a pharmaceutically acceptable salt thereof, is administered at about 1 mg, about 1.5 mg, about 2.0 mg, about 2.5 mg, about 3.0 mg, about 3.5 mg, about 4.0 mg, about 4.5 mg, about 5 mg, about 5.5 mg, or about 6 mg once daily; and the compound of Formula I, or a pharmaceutically acceptable salt thereof, is administered at about 4.0 mg, about 4.5 mg, about 5.0 mg, about 5.5 mg, about 6.0 mg, about 6.5 mg, about 7.0 mg, about 7.5 mg, about 8.0 mg, about 8.5 mg, about 9.0 mg, about 9.5 mg, about 10 mg, about 10.5 mg, about 11 mg, about 12 mg, about 12.5 mg, about 13 mg, about 13.5 mg, about 14 mg, about 14.5 mg, about 15 mg, about 15.5 mg, or about 16 mg once daily.

In embodiments, the compound of Formula I and the compound of Formula III, or a pharmaceutically acceptable salt thereof, are administered simultaneously. In embodiments, the compound of Formula I and the compound of Formula I, or a pharmaceutically acceptable salt thereof, are administered successively.

In embodiments, the compound of Formula III is present in a single dosage form and the compound of Formula I is present in a separate dosage form. In embodiments, the compound of Formula III, the compound of Formula I, or both the compound of Formula III and the compound of Formula I, or pharmaceutically acceptable salt thereof, are administered intravenously, intramuscularly, or orally. In embodiments, the compound of Formula III and the compound of Formula I are comprised in a composition, wherein the composition is an aerosol, an inhalable powder, an injectable, a liquid, a solid, a capsule or a tablet form. In embodiments, the composition further comprises an agent selected from the group consisting of mannitol, microcrystalline cellulose, sodium starch glycolate, sucrose monopalmitate, hydroxypropyl methylcellulose, colloidal silicon dioxide, and sodium stearyl fumarate. In embodiments, the composition further comprises an additive selected from the group consisting of gelatine, titanium dioxide, red iron oxide, and yellow iron oxide.

Also provided herein is a method of treating Childhood-Onset Fluency Disorder (COFD) in a subject in need thereof, comprising administering to a subject in need thereof a therapeutically effective amount of: a compound of the compound of Formula III:

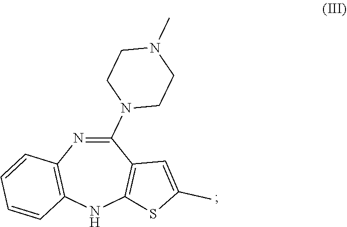 [SMILES: Cc1cc2c(s1)Nc1ccccc1N=C2N1CCN(C)CC1]

and
a crystalline solid of Formula I:

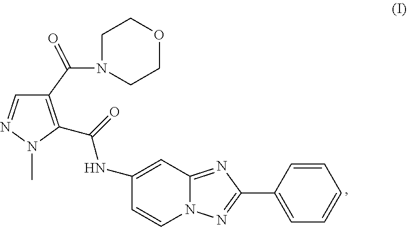 [SMILES: Cn1ncc(C(=O)N2CCOCC2)c1C(=O)Nc1ccn2nc(-c3ccccc3)nc2c1]

wherein said crystalline solid has a melting point onset as determined by DSC of about 210° C. to about 214° C.; or a pharmaceutically acceptable salt thereof.

In some embodiments, the wherein the compound of Formula III, or a pharmaceutically acceptable salt thereof, is administered at about 1 mg to about 500 mg once daily; and the crystalline solid of Formula I, or a pharmaceutically acceptable salt thereof, is administered at about 1 mg to about 500 mg once daily. In some embodiments, the compound of Formula III, or a pharmaceutically acceptable salt thereof, is administered at about 1 mg to about 50 mg once daily; and the crystalline solid of Formula I, or a pharmaceutically acceptable salt thereof, is administered at about 1 mg to about 50 mg once daily. In some embodiments, the compound of Formula III, or a pharmaceutically acceptable salt thereof, is administered at about 1 mg to about 30 mg once daily; and the crystalline solid of Formula I, or a pharmaceutically acceptable salt thereof, is administered at about 500 μg to about 20 mg once daily. In embodiments, the compound of Formula III, or a pharmaceutically acceptable salt thereof, is administered at about 2.5 mg to about 5.0 mg once daily; and the crystalline solid of Formula I, or a pharmaceutically acceptable salt thereof, is administered at about 5.0 mg to about 15 mg once daily. In embodiments, the compound of Formula III, or a pharmaceutically acceptable salt thereof, is administered at about 1 mg, about 1.5 mg, about 2.0 mg, about 2.5 mg, about 3.0 mg, about 3.5 mg, about 4.0 mg, about 4.5 mg, about 5 mg, about 5.5 mg, or about 6 mg once daily; and the crystalline solid of Formula I, or a pharmaceutically acceptable salt thereof, is administered at about 4.0 mg, about 4.5 mg, about 5.0 mg, about 5.5 mg, about 6.0 mg, about 6.5 mg, about 7.0 mg, about 7.5 mg, about 8.0 mg, about 8.5 mg, about 9.0 mg, about 9.5 mg, about 10 mg, about 10.5 mg, about 11 mg, about 12 mg, about 12.5 mg, about 13 mg, about 13.5 mg, about 14 mg, about 14.5 mg, about 15 mg, about 15.5 mg, or about 16 mg once daily.

In some embodiments, the compound of Formula III and the crystalline solid compound of Formula I, or a pharmaceutically acceptable salt thereof, are administered simultaneously. In some embodiments, the compound of Formula I, or a pharmaceutically acceptable salt thereof, are administered successively. In some embodiments, the compound of Formula III is present in a single dosage form and the crystalline solid of Formula I is present in a separate dosage form. In some embodiments, the compound of Formula III, the crystalline solid of Formula I, or both the compound of Formula III and the crystalline solid of Formula I, or pharmaceutically acceptable salt thereof, are administered intravenously, intramuscularly, or orally. In some embodiments, the compound of Formula III and the crystalline solid of Formula I are comprised in a composition, wherein the composition is an aerosol, an inhalable powder, an injectable, a liquid, a solid, a capsule or a tablet form. In some embodiments, the composition further comprises an agent selected from the group consisting of mannitol, microcrystalline cellulose, sodium starch glycolate, sucrose monopalmitate, hydroxypropyl methylcellulose, colloidal silicon dioxide, and sodium stearyl fumarate. In some embodiments, the composition further comprises an additive selected from the group consisting of gelatine, titanium dioxide, red iron oxide, and yellow iron oxide.

Also provided herein is a method of treating Childhood-Onset Fluency Disorder (COFD) in a subject in need thereof, comprising administering to a subject in need thereof a therapeutically effective amount of a compound of Formula III:

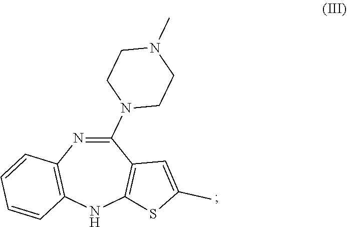 [SMILES: Cc1cc2c(s1)Nc1ccccc1N=C2N1CCN(C)CC1]

and
a compound of Formula I:

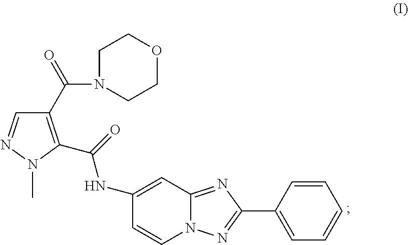 [SMILES: Cn1ncc(C(=O)N2CCOCC2)c1C(=O)Nc1ccn2nc(-c3ccccc3)nc2c1]

or a pharmaceutically acceptable salt thereof, wherein the compound of Formula III, or a pharmaceutically acceptable salt thereof, is administered at about 2.5 mg to about 5.0 mg once daily; and the compound of Formula I, or a pharmaceutically acceptable salt thereof, is administered at about 5.0 mg to about 15 mg once daily.

Also provided herein is a method of treating Childhood-Onset Fluency Disorder (COFD) in a subject in need thereof, comprising administering to a subject in need thereof a therapeutically effective amount of a compound of Formula III:

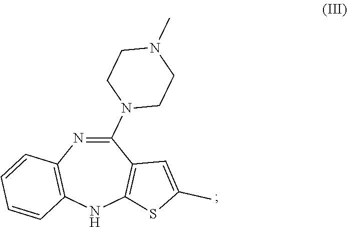 [SMILES: Cc1cc2c(s1)Nc1ccccc1N=C2N1CCN(C)CC1]

and
a crystalline solid of Formula I:

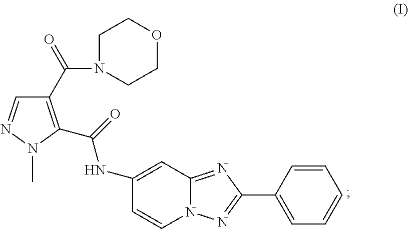 [SMILES: Cn1ncc(C(=O)N2CCOCC2)c1C(=O)Nc1ccn2nc(-c3ccccc3)nc2c1]

or a pharmaceutically acceptable salt thereof, wherein the compound of Formula III, or a pharmaceutically acceptable salt thereof, is administered at about 2.5 mg to about 5.0 mg once daily; and the crystalline solid of Formula I, or a pharmaceutically acceptable salt thereof, is administered at about 5.0 mg to about 15 mg once daily.

Without further elaboration, it is believed that one skilled in the art can, based on the above description, utilize the present invention to its fullest extent. The following specific examples are therefore to be construed as merely illustrative, and not limitative of the remainder of the disclosure in any way whatsoever.

### EXAMPLES

In order that the disclosure described herein may be more fully understood, the following examples are set forth. The examples described in this application are offered to illustrate the compounds, pharmaceutical compositions, and methods provided herein and are not to be construed in any way as limiting their scope.

### Example 1: Synthesis of 1-methyl-4-(morpholine-4-carbonyl)-N-(2-phenyl[1,2,4]triazolo[1,5-a]pyridin-7-yl)-1H-pyrazole-5-carboxamide (Compound of Formula I) [See U.S. Pat. No. 8,349,824]

### 1. 1-methyl-5-(2-phenyl-[1,2,4]triazolo[1,5-a]pyridin-7-ylcarbamoyl)-1H-pyrazole-4-carboxylic acid methyl ester

Step A—1,2-diamino-4-bromo-pyridinium2,4,6-trimethyl-benzenesulfonate: to a cooled suspension of O-(mesitylsulfonyl)hydroxylamine (11.22 g, 52.1 mmol, 1 eq) in dichloromethane (130 ml) was portionwise added 4-bromopyridin-2-amine (9.3 g, 52.1 mmol, 1 eq.) (exothermic reaction, some cooling is needed) giving a white suspension. After 1 hour the white suspension was diluted with diethyl ether (120 ml). The white solid was collected by filtration, washed with diethyl ether and dried affording 1,2-diamino-4-bromo-pyridinium 2,4,6-trimethyl-benzenesulfonate (16.74 g, 82.7%) as white crystals. mp.: 176-180° C. MS: m/z=188.2, 190.2 (M+H+).

Step B—7-bromo-2-phenyl-[1,2,4]triazolo[1,5-a]pyridine: 1,2-Diamino-4-bromopyridinium 2,4,6-trimethylbenzenesulfonate (15.6 g, 40.2 mmole) in pyridine (106 ml) was heated overnight at 100° C., with benzoyl chloride (9.4 ml, 80 mmole) giving a redbrown solution and after 2 hrs a brown suspension. The reaction mixture was concentrated in vacuo and the residue was triturated for 2.5 hr in saturated aqueous ammonium chloride solution (300 ml), while neutralizing to pH 6-7 with saturated aqueous sodium bicarbonate solution. The solid was collected by filtration, washed with water (40 ml) and dried affording 7-bromo-2-phenyl-[1,2,4]triazolo[1,5-a]pyridine (6.78 g, 61.6%) as an off-white solid. mp.: 189-191° C. MS: m/z=276.1, 274.2 (M+H⁺).

Step C—(2-phenyl-[1,2,4]triazolor[1,5-a]pyridin-7-yl)-carbamic acid tert-butyl ester: to an nitrogen purged suspension of 7-bromo-2-phenyl-[1,2,4]triazolo[1,5-a]pyridine (9 g, 32.8 mmol) in dioxane (180 ml) was added successively tert-butyl carbamate (4.71 g, 39.4 mmol), tris(dibenzylidene-acetone)dipalladium(0) (601 mg, 657 μmol), 4,5-bis(diphenylphos-phino)-9,9-dimethylxanthene (760 mg, 1.31 mmol) and cesium carbonate (15 g, 46 mmol). The brown mixture was then stirred for 22 hours at 100° C., under nitrogen atmosphere. The solvent was removed in vacuo and the brown residue partitioned between ethyl acetate and water. The aqueous layer was extracted twice with ethyl acetate and the combined organic layers were washed with water (3×120 ml) and with brine and dried with magnesium sulfate. The solution was concentrated in vacuo to ca 80 ml: crystallization. The suspension was stirred for 10 min in an ice bath and the solid was collected by filtration, washed with little cold ethyl acetate and dried affording (2-phenyl-[1,2,4]triazolo[1,5-a]pyridin-7-yl)-carbamic acid tert-butyl ester (7.09 g) as an off-white solid. The mother liquor was evaporated and the residue (4.79 g) loaded on silica (16 g). The product was isolated by chromatography on a 120 g silica cartridge (eluent heptane/ethyl acetate 10-50%, 45 min) yielding a second crop of 1.748 g of a white solid. mp.: 200-201° C. dec. MS: m/z=311.3 (M+H+). Total yield: 86.7%.

Step D—2-phenyl-[1,2,4]triazolo[1,5-a]pyridin-7-ylamine: a suspension of (2-phenyl-[1,2,4]triazolo[1,5-a]pyridin-7-yl)-carbamic acid tert-butyl ester (8.5 g, 27.4 mmol) in hydrochloric acid (6 N in diethyl ether, 175 ml) was stirred overnight at room temperature. The suspension was diluted under cooling with water (ca 2 l) and ethyl acetate, the aqueous layer was washed once with ethyl acetate, made alkaline with 32% aqueous sodium hydroxide and extracted twice with ethyl acetate. The combined organic layers were dried with magnesium sulfate and the solvent was removed in vacuo affording 2-phenyl-[1,2,4]triazolo[1,5-a]pyridin-7-ylamine (5.52 g, 95.9%) as a light pink solid. mp.: 212-213° C. MS: m/z=211.2 (M+H⁺).

Step E—1-methyl-5-(2-phenyl-[1,2,4]triazolo[1,5-a]pyridin-7-ylcarbamoyl)-1H-pyrazole-4-carboxylic acid methyl ester: a solution of 2-phenyl-[1,2,4]triazolo[1,5-a]pyridin-7-ylamine (1.534 g, 7.3 mmol), 4-(methoxycarbonyl)-1-methyl-1H-pyrazole-5-carboxylic acid (1.61 g, 8.76 mmol), propylphosphonic anhydride (50% in ethyl acetate, 10.7 ml, 18.2 mmol) and diisopropylethylamine (5.1 ml, 29.2 mmol) in tetrahydrofurane (54 ml) was stirred at 70° C., for 1.25 hr giving a white suspension. The cooled suspension was poured in saturated aqueous sodium bicarbonate solution (200 ml), stirred at room temperature for 15 min and the solid was collected by filtration, washed with water and dried affording 1-methyl-5-(2-phenyl-[1,2,4]triazolo[1,5-a]pyridin-7-ylcarbamoyl)-1H-pyrazole-4-carboxylic acid methyl ester (2.596 g, 94.5%) as a white solid. mp.: 243-7° C. MS: m/z=377.2 (M+H⁺).

### 2. 1-Methyl-5-(2-phenyl-[1,2,4]triazolo[1,5-a]pyridin-7-ylcarbamoyl)-1H-pyrazole-4-carboxylic acid

A white suspension of 1-methyl-5-(2-phenyl-[1,2,4]triazolo[1,5-a]pyridin-7-ylcarbamoyl)-1H-pyrazole-4-carboxylic acid methyl ester (2.37 g, 6.3 mmol) and lithium hydroxide monohydrate (291 mg, 6.93 mmol) in methanol (100 ml) and water (20 ml) was stirred for 1.25 hr at 70° C., giving after 20 min a colorless solution. The methanol was removed in vacuo, the residue was diluted with water and the cooled aqueous solution was neutralized with 2N aqueous hydrochloric acid (3.46 ml, 6.03 mmol). The solid was collected by filtration and dried affording 1-methyl-5-(2-phenyl-[1,2,4]triazolo[1,5-a]pyridin-7-ylcarbamoyl)-1H-pyrazole-4-carboxylic acid (2.21 g, 97%) as a white solid. mp.: >300° C. MS: m/z=361.1 (M+H+).

### 3. 1-methyl-4-(morpholine-4-carbonyl)-N-(2-phenyl[1,2,4]triazolo[1,5-a]pyridin-7-yl)-1H-pyrazole-5-carboxamide

A mixture of 1-methyl-5-(2-phenyl-[1,2,4]-triazolo[1,5-a]pyridin-7-ylcarbamoyl)-1H-pyrazole-4-carboxylic acid (100 mg, 276 μmol), morpholine (240 μl, 2.76 mmol) and propylphosphonic anhydride (50% in ethyl acetate, 407 μl, 690 μmol) in tetrahydrofurane (7 ml) was stirred for 3 hours at 70° C. The mixture was diluted with ethyl acetate and washed with saturated aqueous sodium hydrogencarbonate solution and brine. The organic layer was separated, dried with magnesium sulfate and the solvent evaporated. The residue (76 mg white foam) was triturated with diethylether and ethyl acetate affording 1-methyl-4-(morpholine-4-carbonyl)-N-(2-phenyl-[1,2,4]triazolo[1.5-a]pyridin-7-yl)-1H-pyrazole-5-carboxamide (53 mg, 44.5%) as a white solid. mp.: 203-207° C. MS: m/Z=432.4 (M+H″).

### Example 2: A Crystalline Form of Compound of Formula I

Preparation of Crystalline Form: Amorphous form of the compound of Formula I was stirred in water or a mixture of 50% water in methanol at 65° C. for 3 days to afford a crystalline form of the free base of the compound of Formula I. The crystalline form is thermodynamically stable at least between 20° C. and 60° C. The XRPD and DSC graphs were as substantially shown in FIGS. 1 and 2. The XRPD pattern can be obtained by following protocols known in the field.

Melting Point: The maximal melting point peak (Tₘ) of the crystalline form was determined using DSC, which was performed using a Mettler Toledo DSC 821e with Sample Robot TSO 801RO. A sample of 2-5 mg was placed in AL-crucibles 40 uL with AL-piercing lids and the sample was heated at a rate of 10° C./min from 25° C. to 300° C. Temperatures at crystalline melting peak start, peak onset, peak max, and peak end were collected. DSC shows the Tₘ of the crystalline form was 213.16° C.

Solubility: The above crystalline form showed a very low solubility in aqueous solutions at pH>3 (<0.004 mg/mL). Solubility in simulated gastric fluid (SGF), fasted state simulated intestinal fluid (FaSSIF), and fed state simulated intestinal fluid (FeSSIF) was 0.019 mg/ml, 0.006 mg/mL, and 0.022 mg/mL, respectively. Solubility increased 50-100-fold in the presence of surfactants (e.g., Tween-80, sodium dodecyl sulfate, dioctyl sulfosuccinate, or Pluronic F68) and cyclodextrins; however it was still rather low. Overall, the crystalline exhibited a poor solubility at ambient temperature (22° C.) not only in aqueous systems, but also in most of the tested organic solvents (<50 mg/mL).

Stability: Preliminary forced degradation study of the crystalline form of the free base of the compound of Formula I in acidic, basic, oxidative, and photolytic stress conditions was conducted to investigate the viability of the crystalline form in various stress conditions. Since the solubility of the compound of Formula I was low in the standard solvent ethanol, ethanol was replaced by N-methyl-2-pyrrolidone (NMP).

A defined amount of the crystalline form of the free base of the compound of Formula I (0.2-0.8 mg) was weighed into a 1.8 mL HPLC vial and stored open (75% RH) or closed (ambient) at the temperature and the time indicated. After incubation, compound was dissolved in 1-1.5 mL NMP to a final concentration of 0.2-0.5 mg/mL and submitted to UPLC analysis (254 nm). The results are as substantially shown in Table 5 below. The data show that the crystalline form was stable at various temperatures in solid state (<0.5% degradation products), e.g., stable for at least 4 weeks at 40-60° C.

TABLE 5
Preliminary Solid State Stability

| Storage condition | UPLC analysis (Area % main peak) |
|---|---|
| 4 w, 40° C. | 100 |
| 4 w 60° C. | 100 |
| 4 w, 40° C., 75% RH | 100 |
| 4 w, RT | 100 |
| 4 w, 4° C. | 100 |
| 1 h, 80° C. | 100 |
| 1 d, 80° C. | 100 |
| 8 d, 80° C. | 100 |

The crystalline form of the free base of the compound of Formula I was stable for up to 8 days in solid state at 80° C. and in solution at pH 3 to pH 7 at RT. At higher or lower pH, degradation was observed. The crystalline form was stable after exposure to light in solid state. In solution, it was stable towards daylight and, time dependent, moderately stable to unstable in the Suntest. It was also stable towards oxidation for 1 day, but showed some sensitivity upon prolonged exposure. The crystalline form was compatible with almost all excipients; however drug recovery in the CompaS assay strongly depended on the solvent used for extraction.

In the suspension vehicle used for PK, PD, and Tox studies (0.5% HPC/1% Tween 80), The crystalline form was also stable for up to 5 weeks at RT, with only minor particle growth and no hydrate formation. For parenteral administration, a 30% Kleptose formulation (1.5 mg/ml, physiologic osmolality, pH 6.2), with a stability of at least 4 weeks at 40° C. was developed.

### Example 3: A Study of the Safety and Efficacy of the Compound of Formula I for the Treatment of Subjects with COFD

### 1. Study A

Provided below is an open-label, Phase IIa, multi-center, 6-week prospective study to evaluate the safety and efficacy of the compound of Formula I at a daily dose range of 5 mg to 15 mg in adult male patients with COFD.

### Study Design

| | |
|---|---|
| PDE10A | Compound of Formula I (i.e. Compound 1, RO5545965) |
| inhibitor | |
| Dosing | 5 to 15 mg, once daily, oral |
| Efficacy | Primary: Statistically significant improvement on the |
| | Stuttering Severity Instrument-Fourth Edition (SSI-4) |
| | Key secondary: Superior efficacy on the Subject-rated Self |
| | Assessment of Stuttering |
| | Secondary: Superior percent of patients with stuttering |
| | freedom |
| | Secondary: Improvement on patient functioning using the |
| | Sheehan Disability Scale (work, social life, family life |
| | and home responsibilities) |
| Safety | Less than 5% of patients with significant weight gain (greater |
| | than 7% versus baseline); |
| | No or minimal effect on blood glucose and lipids |
| Selection | Number of patients: 24 |
| Criteria | Males aged 18 to 50 years |
| | Meet DSM-5 diagnostic criteria for COFD |
| | Not benefiting from existing therapy |

### 2. Study B

Provided below is a study design of a double-blind, placebo-controlled, Phase IIb, multi-center, six to ten-week prospective study to evaluate the efficacy and safety of the Compound of Formula I (i.e. Compound 1, RO5545965), which is a PDE10A inhibitor, at a daily dose range of 2.5 mg to 15 mg in adult male patients with COFD. The endpoint refers to the first visit following the last dose of study medication.

Overall Design: Following screening to confirm eligibility, on day −7, participants will enter a variable placebo run-in period for up to 5 weeks. Starting from Baseline Visit (Day 1), participants may be randomized to a double-blind, placebo-controlled, parallel-group treatment with Compound of Formula I or placebo, od. During the first 3 weeks of treatment following randomization, participants will receive escalating doses of Compound of Formula I or double-blind escalation of placebo until their maximum tolerated dose is achieved. Thereafter, participants will be maintained at this dose until they have completed a total of 10 weeks treatment period.

Population: An estimated total of 67 male participants age 18 to 50 years who satisfy DSM-5 criteria for Childhood Onset Fluency Disorder (COFD) and require pharmacotherapy and who have a history of stuttering for >2 years with onset consistent to developmental in nature before age 8 years will be enrolled in the study. It is estimated that 60 participants will be randomized to receive either Compound of Formula I or placebo.

Objectives and Endpoints:

| | |
|---|---|
| Objectives | Endpoints |

PRIMARY

| | |
|---|---|
| To evaluate the efficacy of the | Change from baseline to end point |
| compound of Formula I on speech | in severity subset of the Maguire- |
| fluence in adult patients with | Leal-Garibaldi Self-rated |
| COFD. | Stuttering Scale (MLGSSS). |

SECONDARY

| | |
|---|---|
| To evaluate the effect of the | Change from baseline to end |
| compound of Formula I on | point in Sheehan Disability Scale |
| functional impairment. | (SDS). |
| To evaluate the effect of the | Patient global impression of |
| compound of Formula I on | severity (PGI-S) at end point. |
| severity of illness as rated by the | |
| patient. | |
| To evaluate the effect of the | Clinician global impression of |
| compound of Formula I on the | change (CGI-C) at end point. |
| severity of illness as rated by the | |
| clinician. | |
| To evaluate the patient's | Rating of the medication |
| satisfaction in treatment with the | satisfaction questionnaire at end |
| compound of Formula I. | point. |
| To evaluate the effect of the | Change from baseline to end |
| compound of Formula I on the | point in clinician-rated stuttering |
| change in stuttering severity. | severity instrument-4. |

SAFETY

| | |
|---|---|
| To evaluate the safety and | Incidence and severity of adverse |
| tolerability of the compound of | events, plus assessment of |
| Formula I. | hematology, clinical chemistry, |
| | and vital signs. |

Exclusion Criteria: Participants are excluded from the study if any of the following criteria apply:

### Medical Conditions:

1. Stuttering is related to a known neurological cause eg, stroke, etc.
2. Low IQ in the opinion of the investigator.
3. Patients with uncontrolled seizure disorders.
4. A history of severe traumatic brain injury or stroke.
5. Patients who are, in the investigator's opinion, at imminent risk of suicide.
6. Known to have tested positive for human immunodeficiency virus.
7. Known DSM-5 diagnosis of substance abuse or dependence.
8. Unstable medical illness or clinically significant abnormalities on screening tests/exams.
9. Any unstable medical conditions or are currently ill (eg, congenital heart disease, arrhythmia or cancer), which, in the investigator's judgment, will put them at a risk of major adverse event during this trial, are expected to progress during the study, or will interfere with safety and efficacy assessments.

### Prior/Concomitant Therapy:

10. Initiation of new behavioral therapies for stuttering within 10 weeks prior to baseline.

### Prior/Concurrent Clinical Study Experience:

11. Participation in another clinical study with an IP administered in the last 30 days.
12. Participants with a known hypersensitivity to Compound 1 or any of the excipients of the product.

### Diagnostic Assessments:

13. Positive urine drug screen for cocaine or nonprescribed opiates.

### Other Exclusions:

14. Involvement in the planning and/or conduct of the study (applies to both Noema staff and/or staff at the study site).
15. Judgment by the investigator that the participant should not participate in the study if the participant is unlikely to comply with study procedures, restrictions, and requirements.
16. Previous randomization in the present study.

Product Description:

| | |
|---|---|
| Type | Drug |
| Dose formulation | Capsule |
| Unit dose strength(s) | 2.5 mg, 5.0 mg, and 10 mg |
| Dosage level(s) | Once daily 2.5 to 15 mg |
| Route of administration | Oral |

The dose level of Compound of Formula I will be slowly up-titrated from a starting dose of 2.5 mg to a maximum dose of 15 mg, according to the tolerability shown by the individual participant.

### Example 4: A Study of the Effects of the Compound of Formula III (Olanzapine) and the Compound of Formula I (RO5545965) on Glucose Tolerance (Ogtt)

Three consecutive studies were performed to evaluate the effects of Olanzapine (compound of Formula III) and compound of Formula I (RO5545965) on glucose tolerance.

The first objective was to validate healthy rat as predictive model for revealing metabolic side-effects induced by Olanzapine (compound of Formula III). The study was conducted on 10-week male SD rats fed with chow diet. The glucose challenge was performed 1 hour post last treatment. The glucose load was done per os per gavage (2 g/kg BW), and the formulation was made in saline. The details of the formulation are shown in Table 1. The blood glucose and plasma insulin were measured at 0 (before challenge), +15, +30, +60 and +120 minutes. Blood was sampled in EDTA coated tubes, and the blood samples were kept on ice until centrifuged at 4° C. ELISA for Insulin (Mercodia, Uppsala Sweden) and Fluorometric for measuring glucose (AccuCheck System) were used for the analysis. Data Analysis was done using the software JMP for Windows (Version 5.01, SAS institute Inc., SAS Campus Drive, Cary, NC 27513). Analysis of Variance ANOVA (alpha 0.05 and 0.01) followed by Dunnett's test (comparison vs. Control).

TABLE 1
Formulation

| | | | | | | | | | | | | Salt |
|---|---|---|---|---|---|---|---|---|---|---|---|---|
| | | | | | | | | | | Not corrected | | corrected |
| | | | | | | Applications | | | Quantity | Quantity | Compound | Compound |
| | | | Dose | Dose | Animals | per | Duration | BW | (ml) | (ml) | needed (g) | needed (g) |
| Compound | MW | Salt | (mg/kg) | (ml/kg) | (n) | day (n) | (days) | (g) | calcul. | estim. | calculated | calculated |
| Olanzapine | 346.3 | | 3 | 4 | 7 | 1 | 1 | 60 | 1.68 | 2.0 | 0.002 | 0.002 |
| Olanzapine | 346.3 | | 10 | 4 | 7 | 1 | 1 | 60 | 1.68 | 2.0 | 0.005 | 0.005 |
| Haloperidol | 356.4 | | 1 | 4 | 7 | 1 | 1 | 60 | 1.68 | 2.0 | 0.001 | 0.001 |

Vehicle: 0.3% Tween in water.

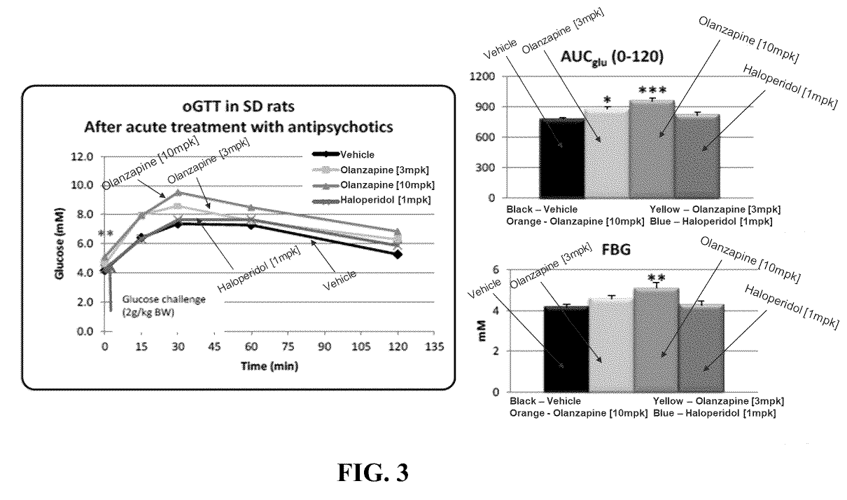

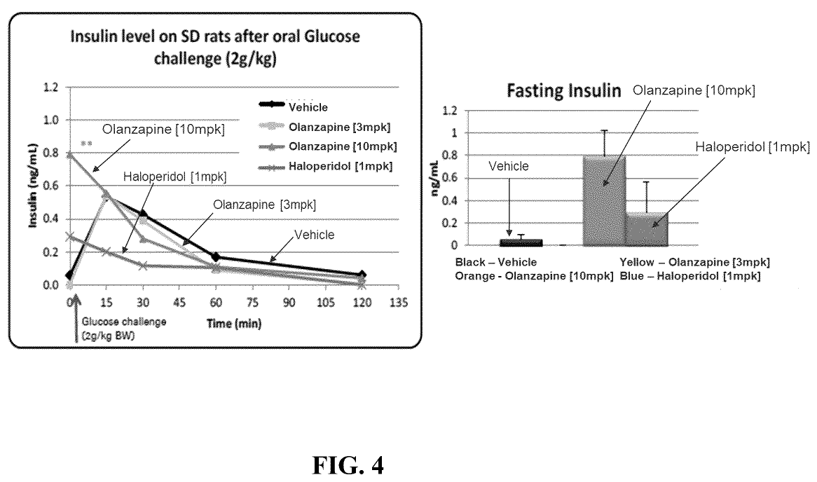

The glucose level during oral glucose tolerance test is shown in FIG. 3, and the insulin levels during oral glucose tolerance test is shown in FIG. 4. The study showed that acute treatment of SD rats with Olanzapine induced a dose-dependent increase of fasting blood glucose, dose-dependent glucose intolerance, and a dose-dependent increase in fasting insulin. Overall, the study revealed that Olanzapine induced glucose intolerance.

The second study assessed the impact of acute treatment with the compound of Formula I. The study was conducted on 9-week male SD rats, which were fed with chow diet. The glucose challenge was performed 1 hour post treatment. Glucose load was done per os per gavage (2 g/kg BW), and the formulation was made in saline. The details of the formulation are shown in Table 2. The blood glucose and plasma insulin were measured at 0 (before challenge), +15, +30, +60 and +120 minutes. Blood was sampled in EDTA coated tubes, and the blood samples were kept on ice until centrifuged at 4° C. ELISA for Insulin (Mercodia, Uppsala Sweden) and Fluorometric for measuring glucose (AccuCheck System) were used for the analysis. Data Analysis was done using the software JMP for Windows (Version 5.01, SAS institute Inc., SAS Campus Drive, Cary, NC 27513). Analysis of Variance ANOVA (alpha 0.05 and 0.01) followed by Dunnett's test (comparison vs. Control).

TABLE 2
Formulation

| | | | | | | Applications | | | Quantity | Quantity | Compound | Calculated |
|---|---|---|---|---|---|---|---|---|---|---|---|---|
| | | | Dose | Dose | Animals | per | Duration | BW | (ml) | (ml) | needed (g) | needed (g) |
| Compound | MW | Salt | (mg/kg) | (ml/kg) | (n) | day (n) | (days) | (g) | calcul. | estim. | calculated | calculated |
| Olanzapine | 346.3 | | 10 | 4 | 8 | 1 | 1 | 250 | 8 | 9.6 | 0.024 | 0.024 |
| RO5545965 | 431.4 | | 10 | 4 | 8 | 1 | 1 | 250 | 8 | 9.6 | 0.024 | 0.024 |
| RO5510629 | 296.3 | | 10 | 4 | 8 | 1 | 1 | 250 | 8 | 9.6 | 0.024 | 0.024 |

Vehicle: 0.3% Tween in water.

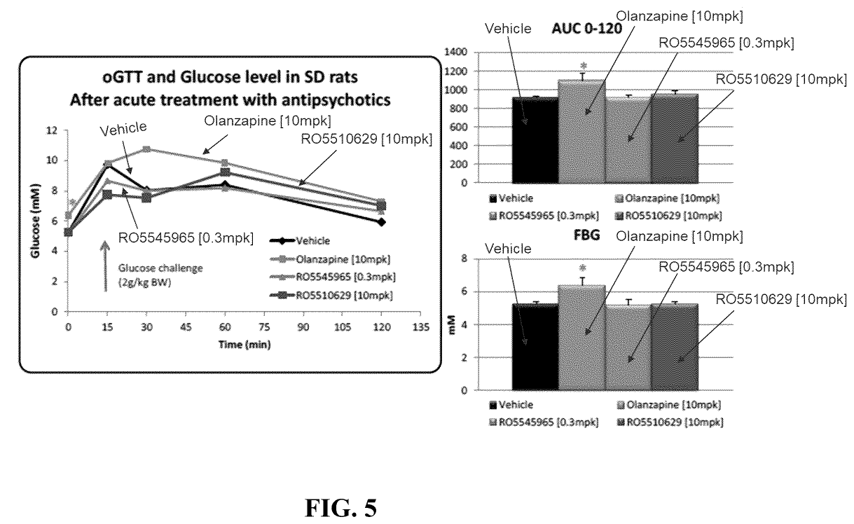

The glucose level during oral glucose tolerance test is shown in FIG. 5. The study showed that an acute treatment of SD rats with Olanzapine induced an increase in fasting blood glucose and glucose intolerance. The details of the formulation are shown in Table 3. The study also showed that acute treatment with the compound of Formula I did not induce any change in glucose tolerance. The third study evaluated sub-chronic (8 days) effects of the compound of Formula I alone or in combination with Olanzapine. The study was conducted on 10-week male SD rats fed with chow diet. The glucose challenge was performed 1 hour post treatment. Glucose load was done per os per gavage (2 g/kg BW (water)). The blood glucose was measured with glucometer at 0 (before challenge), +15, +30, +60 and +120 minutes. The insulin was measured at time 0 only. Blood was sampled in EDTA coated tubes, and the blood samples were kept on ice until centrifuged at 4° C. ELISA for Insulin (Mercodia, Uppsala Sweden) and Fluorometric for measuring glucose (AccuCheck System) were used for the analysis. Data Analysis was done using the software JMP for Windows (Version 5.01, SAS institute Inc., SAS Campus Drive, Cary, NC 27513). Analysis of Variance ANOVA (alpha 0.05 and 0.01) followed by Dunnett's test (comparison vs. Control).

TABLE 3
Formulation

| | | | | | | Applications | | | Quantity | Quantity | Compound | Compound |
|---|---|---|---|---|---|---|---|---|---|---|---|---|
| | | | Dose | Dose | Animals | per | Duration | BW | (ml) | (ml) | needed (g) | needed (g) |
| Compound | MW | Salt | (mg/kg) | (ml/kg) | (n) | day (n) | (days) | (g) | calcul. | estim. | calculated | calculated |
| Olanzapine | 312.4 | | 10 | 4 | 6 | 1 | 1 | 370 | 8.88 | 12.0 | 0.030 | 0.030 |
| RO5545965 | 431.4 | | 0.3 | 4 | 12 | 1 | 8 | 370 | 142.08 | 150.0 | 0.011 | 0.011 |

Vehicle: 0.3% Tween 80 in water.

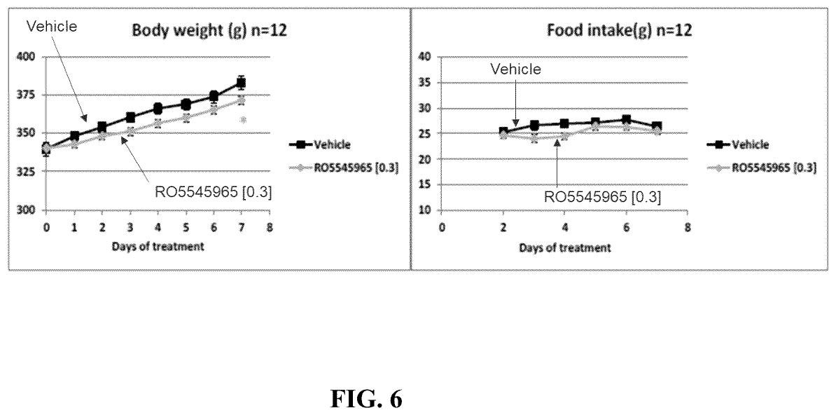

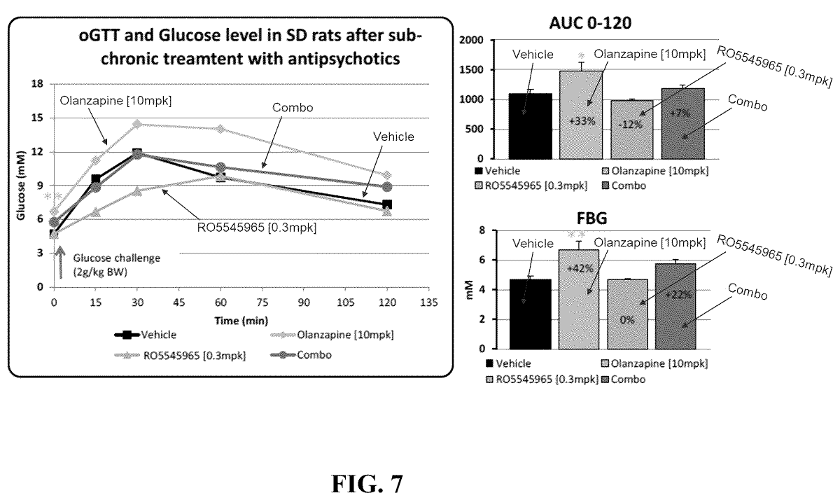

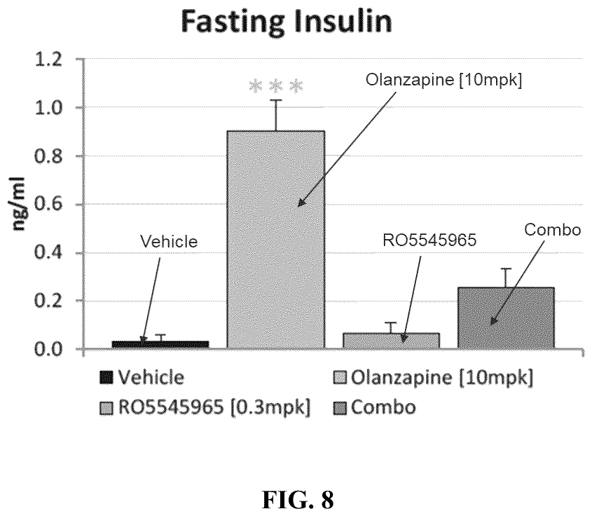

The body weight and food intake is shown in FIG. 6, the glucose level during oral glucose tolerance test is shown in FIG. 7, and the insulin level before oral glucose tolerance test is shown in FIG. 8. The study showed that an acute treatment of SD rats with Olanzapine induced an increase in fasting blood glucose and glucose intolerance, and the effects on glucose and insulin are similar to those reported in the two previous studies performed in similar conditions. A repeated treatment with the compound of Formula I induce a slight glucose tolerance improvement, no change in fasting blood glucose, and decrease of fasting insulin when given in combination with Olanzapine versus Olanzapine alone. The study showed that alone or in combination, the compound of Formula I did not induce glucose intolerance in healthy rats. On the opposite, the study showed a slight improvement glucose tolerance induced by the compound of Formula I alone after 8 days of treatment. The study showed persistence of glucose intolerance induced by Olanzapine.

### Example 5: Compositions Containing Compound 1

Described below is a composition of a 10 mg strength capsule containing the Compound of Formula I (i.e. Compound 1, RO5545965). The capsule is characterized as a reddish brown, opaque, size 1 hard capsule.

| Component^{a} | Amount |
|---|---|
| Fill mass | Actual Weight(mg/capsule) |
| Compound 1 | 10.00 |
| Mannitol | 71.60 |
| Microcrystalline Cellulose | 24.00 |
| Sodium Starch Glycolate | 6.00 |
| Sucrose monopalmitate | 1.20 |
| Hydroxypropylmethylcellulose | 6.00 |
| Colloidal Silicon Dioxide | 0.60 |
| Sodium Stearyl Fumarate | 0.60 |
| Subtotal Weight (fill mass) | 120.00 |

^{a}Purified water (Ph. Eur.) is used for wet granulation; it is essentially removed during processing

Described below is a composition of a 5 mg strength capsule containing the Compound of Formula I (i.e. Compound 1, RO5545965). The capsule is characterized as a reddish brown, opaque, size 1 hard capsule.

| Component^{a} | Amount |
|---|---|
| Fill mass | Actual Weight(mg/capsule) |
| Compound 1 | 5.00 |
| Mannitol | 76.60 |
| Microcrystalline Cellulose | 24.00 |
| Sodium Starch Glycolate | 6.00 |
| Sucrose monopalmitate | 1.20 |
| Hydroxypropylmethylcellulose | 6.00 |
| Colloidal Silicon Dioxide | 0.60 |
| Sodium Stearyl Fumarate | 0.60 |
| Subtotal Weight (fill mass) | 120.00 |

^{a}Purified water (Ph. Eur.) is used for wet granulation; it is essentially removed during processing

Provided below is a 2.5 mg strength capsule containing the Compound of Formula I (i.e. Compound 1, RO5545965). The capsule is characterized as a reddish brown, opaque, size 1 hard capsule.

| Component^{a} | Amount |
|---|---|
| Fill mass | Actual Weight(mg/capsule) |
| Compound 1 | 2.50 |
| Mannitol | 79.10 |
| Microcrystalline Cellulose | 24.00 |
| Sodium Starch Glycolate | 6.00 |
| Sucrose monopalmitate | 1.20 |
| Hydroxypropylmethylcellulose | 6.00 |
| Colloidal Silicon Dioxide | 0.60 |
| Sodium Stearyl Fumarate | 0.60 |
| Subtotal Weight (fill mass) | 120.00 |

^{a}Purified water (Ph. Eur.) is used for wet granulation; it is essentially removed during processing

### INCORPORATION BY REFERENCE

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A method of treating Childhood-Onset Fluency Disorder (COFD), comprising administering to a subject in need thereof a composition containing a therapeutically effective amount of a phosphodiesterase 10A (PDE10A) inhibitor or a pharmaceutically acceptable salt thereof.

2. The method of claim 1, wherein administering comprises administering the PDE10A inhibitor, or a pharmaceutically acceptable salt thereof, once daily.

3. The method of claim 1 or 2, wherein administering comprises administering orally the PDE10A inhibitor, or a pharmaceutically acceptable salt thereof.

4. The method of any one of claims 1-3, wherein administering comprises administering the PDE10A inhibitor, or a pharmaceutically acceptable salt thereof, as a unit dose.

5. The method of any one of claims 1-4, wherein the PDE10A inhibitor is selected from the group consisting of papaverine, PF-02545920, RO5545965, TAK-063, AMG 579, and THPP-1.

6. The method of any one of claims 1-5, wherein the PDE10A inhibitor has no effect on insulin resistance.

7. The method of any one of claims 1-6, wherein the composition comprises the PDE10A inhibitor, or a pharmaceutically acceptable salt thereof, as the sole active agent.

8. The method of any one of claims 1-6, wherein the composition comprises the PDE10A inhibitor, or a pharmaceutically acceptable salt thereof, in combination with another therapeutically active agent.

9. The method of claim 8, wherein the other therapeutically active agent is a dopamine receptor antagonist.

10. The method of claim 9, wherein the other therapeutically active agent is a dopamine receptor D1 (DRD1) antagonist.

11. The method of claim 10, wherein the DRD1 antagonist is ecopipam.

12. The method of claim 9, wherein the other therapeutically active agent is a dopamine receptor D2 (DRD2) antagonist.

13. The method of claim 12, wherein the DRD2 antagonist is Olanzapine, Risperidone, Lurasidone, or Pimozide.

14. The method of any one of claims 1-13, wherein the PDE10A inhibitor, or a pharmaceutically acceptable salt thereof, is administered at about 1 mg to about 100 mg once daily.

15. The method of any one of claims 1-14, wherein the PDE10A inhibitor, or a pharmaceutically acceptable salt thereof, is administered at about 2.5 mg to about 15 mg once daily.

16. The method of any one of claims 1-15, wherein the PDE10A inhibitor, or a pharmaceutically acceptable salt thereof, is administered at about 5 mg to about 15 mg once daily.

17. The method of any one of claims 8-16, wherein the other therapeutically active agent is administered at about 0.1 mg to about 10 mg once daily.

18. The method of any one of claims 8-17, wherein the other therapeutically active agent is administered at about 0.5 mg to about 5 mg once daily.

19. The method of any one of claims 8-17, wherein the other therapeutically active agent is administered at about 2.5 mg to about 5 mg once daily.

20. The method of claim 9, wherein the PDE10A inhibitor, or a pharmaceutically acceptable salt thereof, is administered at about 5 mg to about 15 mg once daily; and the dopamine receptor antagonist is administered at about 0.5 mg to about 5 mg once daily.

21. A method of treating Childhood-Onset Fluency Disorder (COFD), comprising administering to a subject in need thereof a composition containing a therapeutically effective amount of a therapeutic agent or a pharmaceutically acceptable salt thereof, wherein the therapeutic agent is a compound of Formula I.

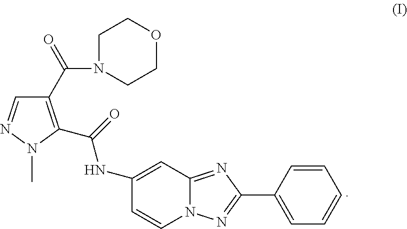 [SMILES: Cn1ncc(C(=O)N2CCOCC2)c1C(=O)Nc1ccn2nc(-c3ccccc3)nc2c1]

22. The method of claim 21, wherein administering comprises administering the compound of Formula I in its free base form.

23. The method of claim 21, wherein administering comprises administering the compound of Formula I in the form of a pharmaceutically acceptable salt thereof.

24. The method of any one of claims 21-23, wherein administering comprises administering orally the PDE10A inhibitor, or a pharmaceutically acceptable salt thereof.

25. The method of any one of claims 21-24, wherein the composition comprises the compound of Formula I, or a pharmaceutically acceptable salt thereof, as the sole active agent.

26. The method of any one of claims 21-24, wherein the composition comprises the compound of Formula I, or a pharmaceutically acceptable salt thereof, in combination with another therapeutically active agent.

27. The method of any one of claims 21-26, wherein the composition comprises inactive agents selected from the group consisting of mannitol, microcrystalline cellulose, sodium starch glycolate, sucrose monopalmitate, hydroxypropyl methylcellulose, colloidal silicon dioxide, and sodium stearyl fumarate.

28. The method of any one of claims 21-27, wherein administering comprises administering the compound of Formula I, or a pharmaceutically acceptable salt thereof, in a capsule.

29. The method of claim 28, wherein the capsule shell consists of gelatine, titanium dioxide, red iron oxide, and yellow iron oxide.

30. The method of claim 29, wherein the capsule comprises about 1 mg to about 10 mg of the compound of Formula I, or a pharmaceutically acceptable salt thereof.

31. The method of claim 29, wherein the capsule comprises about 2.5 mg to about 15 mg of the compound of Formula I, or a pharmaceutically acceptable salt thereof.

32. The method of claim 29, wherein the capsule comprises about 2.5 mg, about 5.0 mg, or about 10 mg of the compound of Formula I, or a pharmaceutically acceptable salt thereof.

33. The method of any one of claims 21-32, wherein administering comprises administering the compound of Formula I, or a pharmaceutically acceptable salt thereof, in an amount of about 5 mg to about 15 mg once daily.

34. The method of any one of claims 21-32, wherein administering comprises administering the compound of Formula I, or a pharmaceutically acceptable salt thereof, in an amount of about 2.5 mg to about 15 mg once daily.

35. The method of any one of claims 21-32, wherein administering comprises administering the compound of Formula I, or a pharmaceutically acceptable salt thereof, in an amount of about 2.5 mg, about 5.0 mg, about 10 mg or 15 mg once daily.

36. A method of treating COFD, comprising administering to a subject in need thereof a composition containing a therapeutically effective amount of a therapeutic agent or a pharmaceutically acceptable salt thereof, wherein the therapeutic agent is a compound of Formula I: 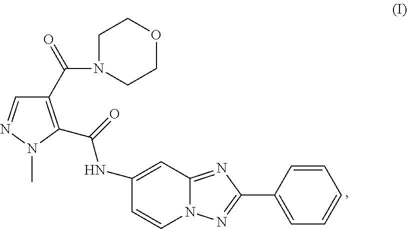 [SMILES: Cn1ncc(C(=O)N2CCOCC2)c1C(=O)Nc1ccn2nc(-c3ccccc3)nc2c1]
wherein administering comprises administering the compound of Formula I, or a pharmaceutically acceptable salt thereof, in an amount of about 5 mg to about 15 mg once daily.

37. A method of treating COFD, comprising administering to a subject in need thereof a composition containing a therapeutically effective amount of a therapeutic agent or a pharmaceutically acceptable salt thereof, wherein the therapeutic agent is a compound of Formula I: 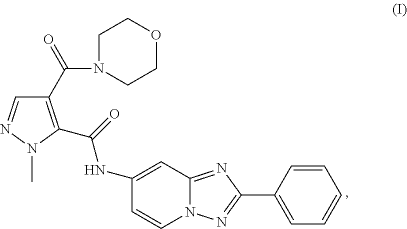 [SMILES: Cn1ncc(C(=O)N2CCOCC2)c1C(=O)Nc1ccn2nc(-c3ccccc3)nc2c1]
wherein administering comprises administering the compound of Formula I, or a pharmaceutically acceptable salt thereof, in an amount of about 2.5 mg to about 15 mg once daily.

38. The method of claim 37, wherein the compound of Formula I, or a pharmaceutically acceptable salt thereof, in an amount of about 2.5 mg, about 5.0 mg, about 10 mg or about 15 mg once daily.

39. A method of treating COFD, comprising administering to a subject in need thereof a composition containing a therapeutically effective amount of a crystalline solid of the compound of Formula I: 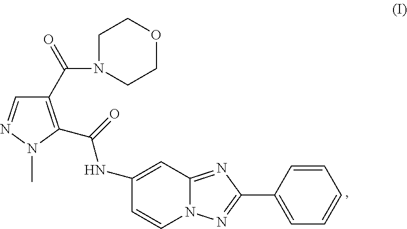 [SMILES: Cn1ncc(C(=O)N2CCOCC2)c1C(=O)Nc1ccn2nc(-c3ccccc3)nc2c1]
wherein said crystalline solid has a melting point onset as determined by DSC of about 210° C. to about 214° C., and said administering comprises administering the crystalline solid in an amount of about 2.5 mg to about 15 mg once daily.

40. The method of claim 39, wherein the administering comprises administering the crystalline solid in an amount of about 5 mg to about 15 mg once daily.

41. The method of claim 39, wherein the administering comprises administering the crystalline solid in an amount of about 2.5 mg, about 5.0 mg, about 10 mg, or about 15 mg once daily.

42. The method of claim 39, wherein the crystalline solid has an XRPD pattern as substantially shown in FIG. 1.

43. The method of claim 39, wherein the crystalline solid has a DSC curve as substantially shown in FIG. 2.

44. The method of any one of claims 1-43, wherein the administering leads to improvement in one or more symptoms of COFD selected from the group consisting of repetition of sounds, repetition of syllables, repetition of words, and prolongation of sounds, blocks and struggle behaviors.

45. A method of treating Childhood-Onset Fluency Disorder (COFD) in a subject in need thereof, comprising administering to a subject in need thereof a therapeutically effective amount of:
a compound of Formula III: 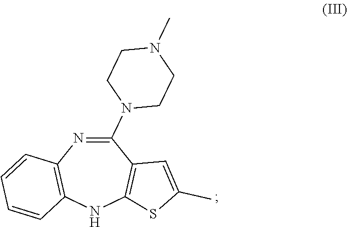 [SMILES: Cc1cc2c(s1)Nc1ccccc1N=C2N1CCN(C)CC1]
and
a compound of Formula I: 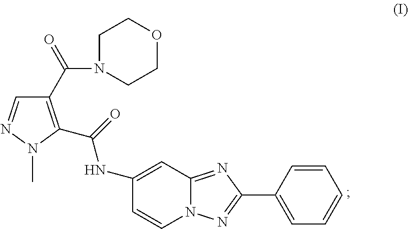 [SMILES: Cn1ncc(C(=O)N2CCOCC2)c1C(=O)Nc1ccn2nc(-c3ccccc3)nc2c1]
or a pharmaceutically acceptable salt thereof.

46. The method of claim 45, wherein the wherein the compound of Formula III, or a pharmaceutically acceptable salt thereof, is administered at about 1 mg to about 500 mg once daily; and the compound of Formula I, or a pharmaceutically acceptable salt thereof, is administered at about 1 mg to about 500 mg once daily.

47. The method of claim 45, wherein the compound of Formula III, or a pharmaceutically acceptable salt thereof, is administered at about 1 mg to about 50 mg once daily; and the compound of Formula I, or a pharmaceutically acceptable salt thereof, is administered at about 1 mg to about 50 mg once daily.

48. The method of claim 45, wherein the compound of Formula III, or a pharmaceutically acceptable salt thereof, is administered at about 1 mg to about 30 mg once daily; and the compound of Formula I, or a pharmaceutically acceptable salt thereof, is administered at about 500 μg to about 20 mg once daily.

49. The method of claim 45, wherein the compound of Formula III, or a pharmaceutically acceptable salt thereof, is administered at about 2.5 mg to about 5.0 mg once daily; and the compound of Formula I, or a pharmaceutically acceptable salt thereof, is administered at about 5.0 mg to about 15 mg once daily.

50. The method of any one of claims 45-49, wherein the compound of Formula III and the compound of Formula I, or a pharmaceutically acceptable salt thereof, are administered simultaneously.

51. The method of any one of claims 45-49, wherein the compound of Formula III and the compound of Formula I, or a pharmaceutically acceptable salt thereof, are administered successively.

52. The method of claim 51, wherein the compound of Formula III is present in a single dosage form and the compound of Formula I is present in a separate dosage form.

53. The method of any one of claims 45-52, wherein the compound of Formula III, the compound of Formula I, or both the compound of Formula III and the compound of Formula I, or pharmaceutically acceptable salt thereof, are administered intravenously, intramuscularly, or orally.

54. The method of any one of claims 45-53, wherein the compound of Formula III and the compound of Formula I are comprised in a composition, wherein the composition is an aerosol, an inhalable powder, an injectable, a liquid, a solid, a capsule or a tablet form.

55. The method of claim 54, wherein the composition further comprises an agent selected from the group consisting of mannitol, microcrystalline cellulose, sodium starch glycolate, sucrose monopalmitate, hydroxypropyl methylcellulose, colloidal silicon dioxide, and sodium stearyl fumarate.

56. The method of claim 55, wherein the composition further comprises an additive selected from the group consisting of gelatine, titanium dioxide, red iron oxide, and yellow iron oxide.

57. A method of treating Childhood-Onset Fluency Disorder (COFD) in a subject in need thereof, comprising administering to a subject in need thereof a therapeutically effective amount of:
a compound of the compound of Formula III: 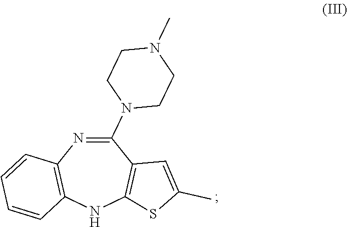 [SMILES: Cc1cc2c(s1)Nc1ccccc1N=C2N1CCN(C)CC1]
and
a crystalline solid of Formula I: 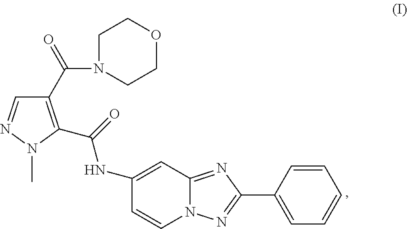 [SMILES: Cn1ncc(C(=O)N2CCOCC2)c1C(=O)Nc1ccn2nc(-c3ccccc3)nc2c1]
wherein said crystalline solid has a melting point onset as determined by DSC of about 210° C. to about 214° C.;
or a pharmaceutically acceptable salt thereof.

58. The method of claim 57, wherein the crystalline solid has an XRPD pattern as substantially shown in FIG. 1.

59. The method of claim 57, wherein the crystalline solid has a DSC curve as substantially shown in FIG. 2.

60. The method of any one of claims 57-59, wherein the wherein the compound of Formula III, or a pharmaceutically acceptable salt thereof, is administered at about 1 mg to about 500 mg once daily; and the compound of Formula I, or a pharmaceutically acceptable salt thereof, is administered at about 1 mg to about 500 mg once daily.

61. The method of any one of claims 57-59, wherein the compound of Formula III, or a pharmaceutically acceptable salt thereof, is administered at about 1 mg to about 50 mg once daily; and the compound of Formula I, or a pharmaceutically acceptable salt thereof, is administered at about 1 mg to about 50 mg once daily.

62. The method of any one of claims 57-59, wherein the compound of Formula III, or a pharmaceutically acceptable salt thereof, is administered at about 1 mg to about 30 mg once daily; and the compound of Formula I, or a pharmaceutically acceptable salt thereof, is administered at about 500 μg to about 20 mg once daily.

63. The method of any one of claims 57-59, wherein the compound of Formula III, or a pharmaceutically acceptable salt thereof, is administered at about 2.5 mg to about 5.0 mg once daily; and the compound of Formula I, or a pharmaceutically acceptable salt thereof, is administered at about 5.0 mg to about 15 mg once daily.

64. The method of any one of claims 57-63, wherein the compound of Formula III and the compound of Formula I, or a pharmaceutically acceptable salt thereof, are administered simultaneously.

65. The method of any one of claims 57-63, wherein the compound of Formula III and the compound of Formula I, or a pharmaceutically acceptable salt thereof, are administered successively.

66. The method of claim 65, wherein the compound of Formula III is present in a single dosage form and the compound of Formula I is present in a separate dosage form.

67. The method of any one of claims 57-66, wherein the compound of Formula III, the compound of Formula I, or both the compound of Formula III and the compound of Formula I, or pharmaceutically acceptable salt thereof, are administered intravenously, intramuscularly, or orally.

68. The method of any one of claims 57-67, wherein the compound of Formula III and the compound of Formula I are comprised in a composition, wherein the composition is an aerosol, an inhalable powder, an injectable, a liquid, a solid, a capsule or a tablet form.

69. The method of claim 68, wherein the composition further comprises an agent selected from the group consisting of mannitol, microcrystalline cellulose, sodium starch glycolate, sucrose monopalmitate, hydroxypropyl methylcellulose, colloidal silicon dioxide, and sodium stearyl fumarate.

70. The method of claim 69, wherein the composition further comprises an additive selected from the group consisting of gelatine, titanium dioxide, red iron oxide, and yellow iron oxide.

71. A method of treating Childhood-Onset Fluency Disorder (COFD) in a subject in need thereof, comprising administering to a subject in need thereof a therapeutically effective amount of:
a compound of Formula III: 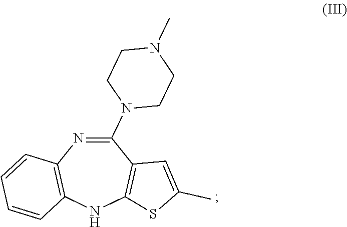 [SMILES: Cc1cc2c(s1)Nc1ccccc1N=C2N1CCN(C)CC1]
and
a compound of Formula I: 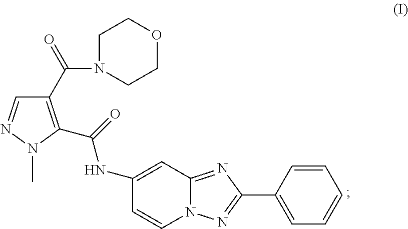 [SMILES: Cn1ncc(C(=O)N2CCOCC2)c1C(=O)Nc1ccn2nc(-c3ccccc3)nc2c1]
or a pharmaceutically acceptable salt thereof, wherein the compound of Formula III, or a pharmaceutically acceptable salt thereof, is administered at about 2.5 mg to about 5.0 mg once daily; and the compound of Formula I, or a pharmaceutically acceptable salt thereof, is administered at about 5.0 mg to about 15 mg once daily.

72. A method of treating Childhood-Onset Fluency Disorder (COFD) in a subject in need thereof, comprising administering to a subject in need thereof a therapeutically effective amount of:
a compound of Formula III: 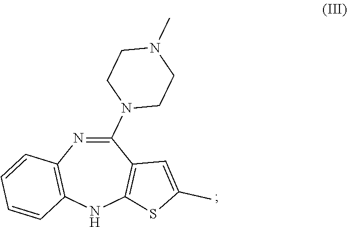 [SMILES: Cc1cc2c(s1)Nc1ccccc1N=C2N1CCN(C)CC1]
and
a crystalline solid of Formula I: 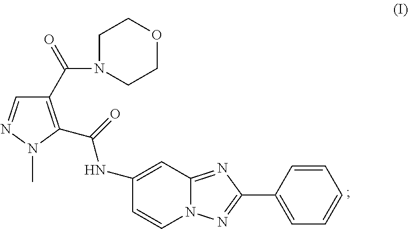 [SMILES: Cn1ncc(C(=O)N2CCOCC2)c1C(=O)Nc1ccn2nc(-c3ccccc3)nc2c1]
or a pharmaceutically acceptable salt thereof, wherein the compound of Formula III, or a pharmaceutically acceptable salt thereof, is administered at about 2.5 mg to about 5.0 mg once daily; and the compound of Formula I, or a pharmaceutically acceptable salt thereof, is administered at about 5.0 mg to about 15 mg once daily.
